(19) United States
(12) Patent Application Publication
(10) Pub. No.: US 2005/0256072 A1
(43) Pub. Date: Nov. 17, 2005

(54) Dual functional oligonucleotides for use in repressing mutant gene expression

(75) Inventors: Neil Aronin, Newtonville, MA (US); Phillip D. Zamore, Northboro, MA (US); Jennifer Broderick, Cambridge, MA (US)

Correspondence Address: LAHIVE & COCKFIELD, LLP., 28 STATE STREET, BOSTON, MA, 02109, US

(73) Assignee: UNIVERSITY OF MASSACHUSETTS, Boston, MA (US)

(21) Appl. No.: 11/055,035

(22) Filed: Feb. 9, 2005

Related U.S. Application Data
(60) Provisional application No. 60/543,467, filed on Feb. 9, 2004.

(51) Int. Cl. A61K 48/00; C12Q 1/68; C07H 21/02; C12N 15/85

(52) U.S. Cl. 514/44; 435/6; 435/455; 536/23.1

(57) ABSTRACT

The present invention is based, in part, on the discovery that endogenous mRNAs can be recruited for translational repression of target mRNAs. The RNA-silencing agents and the methods described herein, thereby provide a means by which to treat genetic (e.g., genetic neurodegenerative diseases such as Huntington's Disease) or non-genetic diseases by, for example, blocking the synthesis of proteins that contribute to the diseases. Accordingly the RNA-silencing agents of the present invention have an mRNA targeting moiety, a linking moiety, and an mRNA recruiting moiety.

### RELATED APPLICATIONS

This application claims priority to U.S. Provisional Application No. 60/543,467, filed Feb. 9, 2004, the entire contents of which are incorporated herein by reference.

### GOVERNMENT INTEREST

This invention was made at least in part with government support under Grant Nos. NS 38194 and R01 GM 062862-04 awarded by the National Institutes of Health. The government may have certain rights in this invention.

### BACKGROUND OF THE INVENTION

RNAi silencing comprises two main approaches to prevent translation of specific proteins: destruction of mRNA (RNA interference, or RNAi) and translational repression. In RNAi, short RNA duplexes (e.g., short interfering RNAs (siRNAs)) destroy specific and complementary mRNAs by cleavage through endonucleases. The short RNA duplexes associate with a protein complex called RNA-induced silencing complex (RISC) to trigger the destruction of the mRNA. In translational repression, endogenous single stranded RNAs (microRNAs (mRNAs)) block the translation of specific and complementary mRNAs. Although, mRNAs also associate with RISC, the association serves simply to repress translation for a brief period while rendering the mRNA intact.

Mammalian cells can produce mRNAs; some mammalian cells have mRNAs in abundance. About one percent of animal genes encode mRNAs, many of which are evolutionally conserved. mRNAs regulate diverse cellular functions, including developmental-timing, cell proliferation, cell death, and fat metabolism. However, the use of mRNA for biological processes in mammals remains elusive. Moreover, the potential of mRNA to affect and control biological processes (e.g., those associated with diseases or disorders) is yet to be harnessed in an effective and efficient manner.

### SUMMARY OF THE INVENTION

The present invention is based, in part, on the discovery that mRNAs can be recruited to block expression of a target mRNA through translational repression. The RNA-silencing agents of the present invention serve to bring endogenous mRNAs within the vicinity of target mRNAs so as to promote the translational repression of the mRNAs.

In one aspect, the invention provides an RNA-silencing agent having the formula T-L-μ, where T is an mRNA targeting moiety, L is a linking moiety, and 1 is a mRNA recruiting moiety. In another aspect, the invention provides an RNA silencing agent suitable for use in repressing translation of a target mRNA, having an mRNA targeting portion complementary to the target mRNA; an mRNA recruiting portion complementary to an mRNA, wherein the mRNA is involved in repressing translation of the target mRNA; and a linking portion that links the mRNA targeting portion and the mRNA recruiting portion.

In one embodiment, the RNA-silencing agent includes an mRNA targeting moiety or portion of about 9 to about 24 nucleotides in length (for example, 15 nucleotides in length). In another embodiment, the RNA-silencing agent includes an mRNA recruiting moiety or portion that is about 13 to about 21 nucleotides in length (for example, about 13 or about 15 nucleotides in length).

In one embodiment, the RNA is silenced via translational repression of the target mRNA. In another embodiment, the mRNA targeting moiety or portion targets an mRNA encoding a protein involved in a disease (e.g., Huntington's Disease) or disorder. In yet another embodiment, the mRNA targeting moiety or portion targets an mRNA encoding huntingtin protein (e.g., mutant huntingtin protein).

In yet another embodiment, the mRNA targeting moiety targets an mRNA encoding a protein (e.g., a mutant protein) selected from the group consisting of matrix metalloproteinase 1, matrix metalloproteinase 2, matrix metalloproteinase 9, metalloelastase, CD36 receptor, tenascin-C, secreted protein acidic and rich in cysteine (SPARC), and androgen receptor gene. Without wishing to be bound to any particular theory, it is believed that these proteins may be involved in cellular proliferative disorders.

In one embodiment, the linking moiety or portion is a phosphodiester bond. In one embodiment, the linking moiety or portion includes at least one modified nucleotide which increases the in vivo stability of the agent. For example, the linking moiety or portion has at least one 2′-O-methyl nucleotide and/or at least one phosphorothioate nucleotide. In another embodiment, the linking moiety or portion has at least one locked nucleotide (e.g., C2′-O,C4′-ethylene-bridged nucleotide). In other embodiments, the linking moiety or portion has at least one sugar-modified nucleotide and/or at least one base-modified nucleotide.

In another embodiment, the mRNA recruiting moiety or portion recruits an mRNA capable of inducing silencing via an RNA induced silencing complex (RISC). In another embodiment, the mRNA recruiting moiety or portion recruits an mRNA selected from Table 1. In yet another embodiment, the mRNA recruiting moiety or portion recruits a let-7 mRNA, a miR124a mRNA, or a miR166 mRNA.

In yet another embodiment, the invention provides a composition including an RNA-silencing agent and a pharmaceutically acceptable carrier.

In another aspect, the invention provides a method of repressing gene (e.g., a gene encoding a protein, for example, a mutant protein such as huntingtin, associated with a disease or a disorder) expression in a cell, including contacting a cell with an RNA-silencing agent, under conditions such that the agent represses gene expression within the cell (e.g., in an organism).

In yet another aspect, the invention provides a method for treating a subject having or at risk for a disease or disorder characterized or caused by the overexpression or overactivity of a cellular protein, including administering to the subject an effective amount of an RNA-silencing agent, wherein the mRNA targeting moiety targets an mRNA encoding said protein.

In yet another aspect, the invention provides a method for treating a subject having or at risk for a disease (e.g., Huntington's Disease) or disorder characterized or caused by the expression or activity of a mutant protein, including administering to the subject an effective amount of an RNA-silencing agent, wherein the mRNA targeting moiety targets an mRNA encoding said protein. In one embodiment, the disease or disorder is characterized or caused by a gain-of-function mutant protein.

In another aspect, the invention provides for the use of an RNA-silencing agent in the manufacture of a medicament for repressing mutant gene expression.

### BRIEF DESCRIPTION OF THE DRAWINGS

FIG. 1 depicts a model of the RNAi pathway mediated by both mRNA and siRNA.

FIG. 2 depicts the recruitment of an endogenous mRNA using the RNA-silencing agents of the present invention. FIG. 2A depicts an RNA-silencing agent and an mRNA, let-7, associated with the protein complex, RISC. FIG. 2B depicts the RNA-silencing agent associating with the target mRNA, luciferase, and the mRNA, let-7.

FIGS. 3A-3B depict the amino acid sequence of the human huntingtin protein (SEQ ID NO:1).

FIGS. 4A-4K depict the nucleotide sequence of the human huntingtin gene (cDNA) (SEQ ID NO:2).

FIG. 5 depicts a model of translational repression mediated by an RNA-silencing agent of the present invention.

FIG. 6 depicts translational repression of Renilla luciferase mRNA in HeLa cells upon binding of 5 nM siRNA with perfect or imperfect (bulged) complementarity to CXCR4 binding site.

FIG. 7 depicts the sequences of transcripts utilized in the exemplification of the present invention.

FIG. 8 depicts the effect of 2′-O-methyl oligonucleotide RNA silencing agents on Renilla luciferase expression.

FIG. 9 depicts the effect of 2′-O-methyl oligonucleotide RNA silencing agents on Renilla luciferase expression.

FIG. 10 depicts Renilla luciferase expression from HeLa cells transfected with pRL-TK reporter vectors containing six target sites for the 2′-O-methyl oligonucleotide miR166/CXCR4 tether.

FIG. 11 depicts the effect of 2′-O-methyl oligonucleotide tethers and miR166 on Renilla luciferase expression from reporter vector pRL-TK containing six target sites for the tether.

FIG. 12 depicts the percent Renilla luciferase expression in HeLa cells cotransfected with reporter vectors pRL-TK and pGL2 with 2′-O-methyl oligonucleotide tether with complementarity to the CXCR4 target site and with homology to antisense miR166.

FIGS. 13 and 14 graphically depict the results of truncations of the mRNA targeting moiety on translational repression of Renilla luciferase expression.

FIGS. 15A and 15B graphically depict the results of truncations of the mRNA recruiting moiety on translational repression of Renilla luciferase expression.

FIG. 16 graphically depicts the results of translational repression mediated by an RNA-silencing agent designed to target let-7 mRNA in accordance with the present invention.

### DETAILED DESCRIPTION OF THE INVENTION

The present invention is based, in part, on the discovery that endogenous mRNAs can be recruited for translational repression of target mRNAs. Accordingly, RNA-silencing agents having an mRNA targeting moiety or portion, a linking moiety or portion, and an mRNA recruiting moiety or portion, are designed to promote mRNA-mediated repression of a target mRNA. The RNA-silencing agents and the methods described herein, thereby provide a means to treat genetic (e.g., genetic neurodegenerative diseases such as Huntington's Disease) or non-genetic diseases by, for example, blocking the synthesis of proteins that contribute to the diseases.

The methods of the present invention offer several advantages over existing techniques to repress the expression of a particular gene. First, the methods described herein allow an endogenous molecule (often present in abundance), an mRNA, to mediate RNA silencing; accordingly the methods described herein obviate the need to introduce foreign molecules (e.g., siRNAs) to mediate RNA silencing, although exogenous mRNAs may be introduced in accordance with the methods of the present invention. Second, the RNA-silencing agents and, in particular, the linking moiety (e.g., oligonucleotides such as the 2′-O-methyl oligonucleotide), can be made stable and resistant to nuclease activity. As a result, the RNA-silencing agents of the present invention can be designed for direct delivery, obviating the need for indirect delivery (e.g., viral) of a precursor molecule or plasmid designed to make the desired agent within the cell. Third, RNA-silencing agents, and their respective moieties, can be designed to conform to specific mRNA sites and specific mRNAs. The designs can be cell and gene product specific. Accordingly, RNA-silencing agents designed in accordance with the present invention can serve to selectively target particular genes in particular tissues for translational repression. Fourth, the methods disclosed herein leave the mRNA intact, allowing one skilled in the art to block protein synthesis in short pulses using the cell's own machinery. As a result, these methods of RNA silencing are highly regulatable.

Definitions

So that the invention may be more readily understood, certain terms are first defined.

As used herein, the term “RNA-silencing agent” refers to a molecule having the formula T-L-μ, wherein T is an mRNA targeting moiety, L is a linking moiety, and μ is an mRNA recruiting moiety.

As used herein, the terms “mRNA targeting moiety”, “targeting moiety”, “mRNA targeting portion” or “targeting portion” refer to a domain, portion or region of the RNA-silencing agent having sufficient size and sufficient complementarity to a portion or region of an mRNA chosen or targeted for silencing (i.e., the moiety has a sequence sufficient to capture the target mRNA).

As used herein, the terms “mRNA recruiting moiety”, “recruiting moiety”, “mRNA recruiting portion” or “recruiting portion” refer to a domain, portion or region of the RNA-silencing agent having a sufficient size and sufficient complementarity to mRNA (e.g., an endogenous cellular mRNA), or portion or region of said mRNA (i.e., the moiety has a sequence sufficient to recruit mRNA).

As used herein, the term “linking moiety” or “linking portion” refers to a domain, portion or region of the RNA-silencing agent which covalently joins or links the mRNA targeting moiety and the mRNA recruiting moiety.

The term “nucleoside” refers to a molecule having a purine or pyrimidine base covalently linked to a ribose or deoxyribose sugar. Exemplary nucleosides include adenosine, guanosine, cytidine, uridine and thymidine. The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

The term “RNA” or “RNA molecule” or “ribonucleic acid molecule” refers to a polymer of ribonucleotides. The term “DNA” or “DNA molecule” or “deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

As used herein, the term “microRNA” (“mRNA”) refers to an RNA (or RNA analog) comprising less than about 25 nucleotides which is capable of directing or mediating translational repression.

The term “nucleotide analog”, also referred to herein as an “altered nucleotide” or “modified nucleotide” refers to a non-standard nucleotide, including non-naturally occurring ribonucleotides or deoxyribonucleotides. Preferred nucleotide analogs are modified at any position so as to alter certain chemical properties of the nucleotide while retaining the ability of the nucleotide analog to perform its intended function.

The term “oligonucleotide” refers to a short polymer of nucleotides and/or nucleotide analogs. The term “RNA analog” refers to a polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. The oligonucleotides may be linked with linkages which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, and/or phosphorothioate linkages. Exemplary RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate (mediates) RNA silencing.

As used herein, the term “translational repression” refers to a type of RNAi silencing in which mRNA mediates the blocking of mRNA translation. Translational repression occurs in cells naturally. Alternatively, translational repression can be initiated by the hand of man, for example, to silence the translation of target genes.

As used herein, the terms “sufficient complementarity” or “sufficient degree of complementarity” mean that the mRNA targeting moiety or the mRNA recruiting moiety has a sequence sufficient to bind the desired mRNA or mRNA, respectively, and to trigger the translational repression of the mRNA.

The term “mismatch” refers to a basepair consisting of noncomplementary bases, for example, not normal complementary G:C, A:T or A:U base pairs.

As used herein, the term “isolated” molecule (e.g., isolated nucleic acid molecule) refers to molecules which are substantially free of other cellular material, or culture medium when produced by recombinant techniques, or substantially free of chemical precursors or other chemicals when chemically synthesized.

A target mRNA refers to an mRNA (e.g., associated with a disease or disorder) to which the mRNA targeting moiety is complementary and for which translational repression is desirable.

A target gene is a gene targeted by an RNA-silencing agent. The mRNA targeting moiety is complementary (e.g., fully complementary) to a section of the mRNA of the target gene.

A gene or mRNA “involved” in a disease or disorder includes a gene or an mRNA, the normal or aberrant expression or function of which effects or causes a disease or disorder or at least one symptom of said disease or disorder.

As used herein, the term “treatment” or “treating” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the infection and the general state of the patient's own immune system.

The term “patient” includes human and other mammalian subjects that receive either prophylactic or therapeutic treatment.

RNA-Silencing Agents

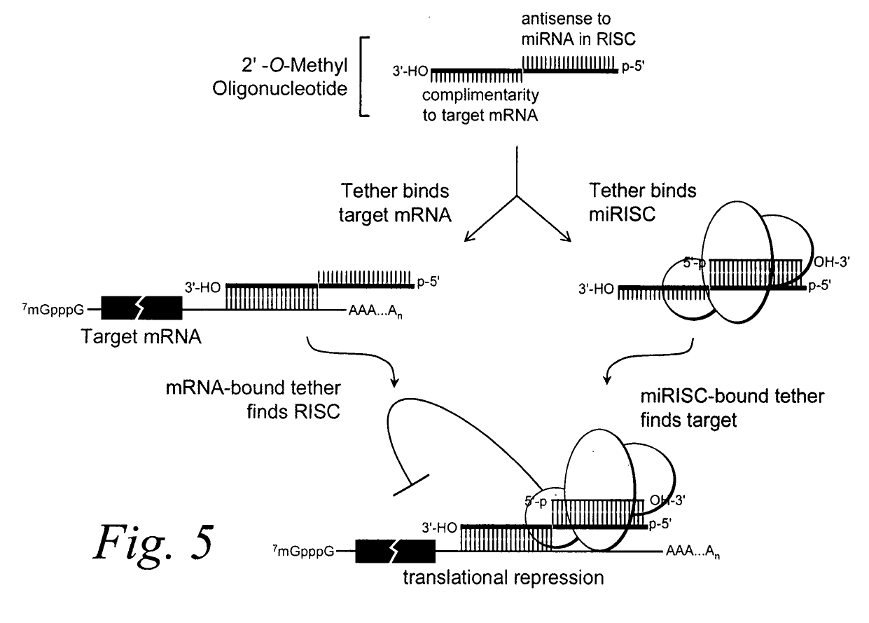

The present invention relates to RNA-silencing agents. The RNA-silencing agents of the invention are designed such that they recruit mRNAs (e.g., endogenous cellular mRNAs) to a target mRNA so as to induce RNA silencing (as shown in FIG. 5). In preferred embodiments, the RNA-silencing agents have the formula T-L-μ, wherein T is an mRNA targeting moiety, L is a linking moiety, and μ is an mRNA recruiting moiety. Any one or more moiety may be double stranded. Preferably, however, each moiety is single stranded.

Moieties within the RNA-silencing agents can be arranged or linked (in the 5′ to 3′ direction) as depicted in the formula T-L-μ (i.e., the 3′ end of the targeting moiety linked to the 5′ end of the linking moiety and the 3′ end of the linking moiety linked to the 5′ end of the mRNA recruiting moiety). Alternatively, the moeities can be arranged or linked in the RNA-silencing agent as follows: μ-T-L (i.e., the 3′ end of the mRNA recruiting moiety linked to the 5′ end of the linking moiety and the 3′ end of the linking moiety linked to the 5′ end of the targeting moiety).

The mRNA targeting moiety, as described above, is capable of capturing a specific target mRNA. According to the invention, expression of the mRNA is undesirable, and, thus, translational repression of the mRNA is desired. In one embodiment, the mRNA encodes a protein involved in a disease or a disorder. For example, the mRNA may encode for huntingtin protein (e.g. mutant huntingtin protein), which is associated with Huntington's disease (a genetic neurodegenerative disease).

The mRNA targeting moiety should be of sufficient size to effectively bind the target mRNA. The length of the targeting moiety will vary greatly depending, in part, on the length of the target mRNA and the degree of complementarity between the target mRNA and the targeting moiety. In various embodiments, the targeting moiety is less than about 200, 100, 50, 30, 25, 20, 19, 18, 17, 16, 15, 14, 13, 12, 11, 10, 9, 8, 7, 6, or 5 nucleotides in length. In a particular one embodiment, the targeting moiety is about 15 to about 25 nucleotides in length. In another embodiment, the targeting moiety is about 9, 10, 11, 12, 13 or 14 to about 24 nucleotides in length. In a particular embodiment, the targeting moiety is about 15 nucleotides in length, e.g., 15, 16, 17 or 18 nucleotides in length.

The mRNA recruiting moiety, as described above, is capable of associating with an mRNA. According to the invention, the mRNA may be any mRNA capable of repressing the target mRNA. Mammals are reported to have over 250 endogenous mRNAs (Lagos-Quintana et al. (2002) Current Biol. 12:735-739; Lagos-Quintana et al. (2001) Science 294:858-862; and Lim et al. (2003) Science 299:1540). In various embodiments, the mRNA may be any of art-recognized mRNA. Table 1 lists some of the known human mRNAs.

TABLE 1
Human miRNAs

| | | | | | |
|---|---|---|---|---|---|
| ID | Species | Gene | miRNA sequence | Mature | Precursor |

| | | | | | |
|---|---|---|---|---|---|
| hsa-mir-7-1 | Homo | miR-7-1 | uggaagacuagugauuuuguu | 21 | 110 |
| | sapiens | | | | |
| hsa-mir-7-2 | Homo | miR-7-2 | uggaagacuagugauuuuguu | 21 | 110 |
| | sapiens | | | | |
| hsa-mir-7-3 | Homo | miR-7-3 | uggaagacuagugauuuuguu | 21 | 110 |
| | sapiens | | | | |
| hsa-let-7f-2L | Homo | let-7f-2 | ugagguaguagauuguauaguu | 22 | 89 |
| | sapiens | | | | |
| hsa-let-7f-1L | Homo | let-7f-1 | ugagguaguagauuguauaguu | 22 | 87 |
| | sapiens | | | | |
| hsa-let-7eL | Homo | let-7e | ugagguaggagguuguauagu | 21 | 79 |
| | sapiens | | | | |
| hsa-let-7a-1L | Homo | let-7a-1 | ugagguaguagguuguauaguu | 22 | 80 |
| | sapiens | | | | |
| hsa-let-7a-2L | Homo | let-7a-2 | ugagguaguagguuguauaguu | 22 | 72 |
| | sapiens | | | | |
| hsa-let-7a-3L | Homo | let-7a-3 | ugagguaguagguuguauaguu | 22 | 74 |
| | sapiens | | | | |
| hsa-let-7bL | Homo | let-7b | ugagguaguagguugugugguu | 22 | 83 |
| | sapiens | | | | |
| hsa-let-7cL | Homo | let-7c | ugagguaguagguuguaugguu | 22 | 84 |
| | sapiens | | | | |
| hsa-let-7dL | Homo | let-7d | agagguaguagguugcauagu | 21 | 87 |
| | sapiens | | | | |
| hsa-mir-10a | Homo | mir-10a | uacccuguagauccgaauuugug | 23 | 110 |
| | sapiens | | | | |
| hsa-mir-10b | Homo | mir-10b | uacccuguagaaccgaauuugu | 22 | 110 |
| | sapiens | | | | |
| hsa-mir-15 | Homo | mir-15 | uagcagcacauaaugguuugug | 22 | 83 |
| | sapiens | | | | |
| hsa-mir-16 | Homo | mir-16 | uagcagcacguaaauauuggcg | 22 | 89 |
| | sapiens | | | | |
| hsa-mir-17 | Homo | mir-17 | acugcagugaaggcacuugu | 20 | 84 |
| | sapiens | | | | |
| hsa-mir-18 | Homo | mir-18 | uaaggugcaucuagugcagaua | 22 | 71 |
| | sapiens | | | | |
| hsa-mir-19a | Homo | mir-19a | ugugcaaaucuaugcaaaacuga | 23 | 82 |
| | sapiens | | | | |
| hsa-mir-19b-1 | Homo | mir-19b-1 | ugugcaaauccaugcaaaacuga | 23 | 87 |
| | sapiens | | | | |
| hsa-mir-19b-2 | Homo | mir-19b-2 | ugugcaaauccaugcaaaacuga | 23 | 96 |
| | sapiens | | | | |
| hsa-mir-20 | Homo | mir-20 | uaaagugcuuauagugcaggua | 22 | 71 |
| | sapiens | | | | |
| hsa-mir-21 | Homo | mir-21 | uagcuuaucagacugauguuga | 22 | 72 |
| | sapiens | | | | |
| hsa-mir-22 | Homo | mir-22 | aagcugccaguugaagaacugu | 22 | 85 |
| | sapiens | | | | |
| hsa-mir-23 | Homo | mir-23 | aucacauugccagggauuucc | 21 | 73 |
| | sapiens | | | | |
| hsa-mir-24-2 | Homo | mir-24-2 | uggcucaguucagcaggaacag | 22 | 73 |
| | sapiens | | | | |
| hsa-mir-24-1 | Homo | mir-24-1 | uggcucaguucagcaggaacag | 22 | 68 |
| | sapiens | | | | |
| hsa-mir-25 | Homo | mir-25 | cauugcacuugucucggucuga | 22 | 84 |
| | sapiens | | | | |
| hsa-mir-26a | Homo | mir-26a | uucaaguaauccaggauaggcu | 22 | 75 |
| | sapiens | | | | |
| hsa-mir-26b | Homo | mir-26b | uucaaguaauucaggauaggu | 21 | 77 |
| | sapiens | | | | |
| hsa-mir-27 | Homo | mir-27 | uucacaguggcuaaguuccgcc | 22 | 78 |
| | sapiens | | | | |
| hsa-mir-28 | Homo | mir-28 | aaggagcucacagucuauugag | 22 | 86 |
| | sapiens | | | | |
| hsa-mir-29 | Homo | mir-29 | cuagcaccaucugaaaucgguu | 22 | 64 |
| | | sapiens | | | |
| hsa-mir-30c | Homo | mir-30c | uguaaacauccuacacucucagc | 23 | 72 |
| | sapiens | | | | |
| hsa-mir-30d | Homo | mir-30d | uguaaacauccccgacuggaag | 22 | 70 |
| | sapiens | | | | |
| hsa-mir-30a | Homo | mir-30a-s | uguaaacauccucgacuggaagc | 23 | 71 |
| | sapiens | | | | |

The mature sequences miR-30 and miR-97 appear to originate from the same
precursor and the entries have been merged and renamed to match the
homologous mouse entry.

| | | | | | |
|---|---|---|---|---|---|
| hsa-mir-30a | Homo | mir-30a-as | cuuucagucggauguuugcagc | 22 | 71 |
| | sapiens | | | | |
| hsa-mir-31 | Homo | mir-31 | ggcaagaugcuggcauagcug | 21 | 71 |
| | sapiens | | | | |
| hsa-mir-32 | Homo | mir-32 | uauugcacauuacuaaguugc | 21 | 70 |
| | sapiens | | | | |
| hsa-mir-33 | Homo | mir-33 | gugcauuguaguugcauug | 19 | 69 |
| | sapiens | | | | |
| hsa-mir-34 | Homo | mir-34 | uggcagugucuuagcugguugu | 22 | 110 |
| | sapiens | | | | |
| hsa-mir-91 | Homo | mir-91 | caaagugcuuacagugcagguagu | 24 | 82 |
| | sapiens | | | | |
| — | Homo | mir-17 | acugcagugaaggcacuugu | 20 | 82 |
| | sapiens | | | | |

miR-17 is cleaved from the reverse strand of human precursor mir-91 and
from human precursor mir-17

| | | | | | |
|---|---|---|---|---|---|
| hsa-mir-92-1 | Homo | mir-92-1 | uauugcacuugucccggccugu | 22 | 78 |
| | sapiens | | | | |
| hsa-mir-92-2 | Homo | mir-92-2 | uauugcacuugucccggccugu | 22 | 75 |
| | sapiens | | | | |
| hsa-mir-93-1 | Homo | mir-93-1 | aaagugcuguucgugcagguag | 22 | 80 |
| | sapiens | | | | |
| hsa-mir-93-2 | Homo | mir-93-2 | aaagugcuguucgugcagguag | 22 | 80 |
| | sapiens | | | | |
| hsa-mir-95 | Homo | mir-95 | uucaacggguauuuauugagca | 22 | 81 |
| | sapiens | | | | |
| hsa-mir-96 | Homo | mir-96 | uuuggcacuagcacauuuuugc | 22 | 78 |
| | sapiens | | | | |
| hsa-mir-98 | Homo | mir-98 | ugagguaguaaguuguauuguu | 22 | 80 |
| | sapiens | | | | |
| hsa-mir-99 | Homo | mir-99 | aacccguagauccgaucuugug | 22 | 81 |
| | sapiens | | | | |
| hsa-mir-100 | Homo | mir-100 | aacccguagauccgaacuugug | 22 | 80 |
| | sapiens | | | | |
| hsa-mir-101 | Homo | mir-101 | uacaguacugugauaacugaag | 22 | 75 |
| | sapiens | | | | |
| hsa-mir-102-1 | Homo | mir-102-1 | uagcaccauuugaaaucagu | 20 | 81 |
| | sapiens | | | | |
| hsa-mir-102-2 | Homo | mir-102-2 | uagcaccauuugaaaucagu | 20 | 81 |
| | sapiens | | | | |
| hsa-mir-102-3 | Homo | mir-102-3 | uagcaccauuugaaaucagu | 20 | 81 |
| | sapiens | | | | |
| hsa-mir-103-2 | Homo | mir-103-2 | agcaacauuguacagggcuauga | 23 | 78 |
| | sapiens | | | | |
| hsa-mir-103-1 | Homo | mir-103-1 | agcagcauuguacagggcuauga | 23 | 78 |
| | sapiens | | | | |
| hsa-mir-104 | Homo | mir-104 | ucaacaucagucugauaagcua | 22 | 78 |
| | sapiens | | | | |
| hsa-mir-105-1 | Homo | mir-105-1 | ucaaaugcucagacuccugu | 20 | 81 |
| | sapiens | | | | |
| hsa-mir-105-2 | Homo | mir-105-2 | ucaaaugcucagacuccugu | 20 | 81 |
| | sapiens | | | | |
| hsa-mir-106 | Homo | mir-106 | aaaagugcuuacagugcagguagc | 24 | 81 |
| | sapiens | | | | |
| hsa-mir-107 | Homo | mir-107 | agcagcauuguacagggcuauca | 23 | 81 |
| | sapiens | | | | |
| hsa-mir-124b | Homo | mir-124b | uuaaggcacgcggugaaugc | 20 | 67 |
| | sapiens | | | | |
| hsa-mir-139 | Homo | mir-139 | ucuacagugcacgugucu | 18 | 68 |
| | sapiens | | | | |
| hsa-mir-147 | Homo | mir-147 | guguguggaaaugcuucugc | 20 | 72 |
| | sapiens | | | | |
| hsa-mir-148 | Homo | mir-148 | ucagugcacuacagaacuuugu | 22 | 68 |
| | sapiens | | | | |
| hsa-mir-181c | Homo | mir-181c | aacauucaaccugucggugagu | 22 | 110 |
| | sapiens | | | | |
| hsa-mir-181b | Homo | mir-181b | accaucgaccguugauuguacc | 22 | 110 |
| | sapiens | | | | |
| hsa-mir-181a | Homo | mir-181a | aacauucaacgcugucggugagu | 23 | 110 |
| | sapiens | | | | |
| hsa-mir-182-as | Homo | mir-182-as | ugguucuagacuugccaacua | 21 | 110 |
| | sapiens | | | | |
| hsa-mir-183 | Homo | mir-183 | uauggcacugguagaauucacug | 23 | 110 |
| | sapiens | | | | |
| hsa-mir-187 | Homo | mir-187 | ucgugucuuguguugcagccg | 21 | 110 |
| | sapiens | | | | |
| hsa-mir-192 | Homo | mir-192 | cugaccuaugaauugacagcc | 21 | 110 |
| | sapiens | | | | |
| hsa-mir-196-2 | Homo | mir-196-2 | uagguaguuucauguuguuggg | 22 | 110 |
| | sapiens | | | | |
| hsa-mir-196-1 | Homo | mir-196-1 | uagguaguuucauguuguuggg | 22 | 110 |
| | sapiens | | | | |
| hsa-mir-196 | Homo | mir-196 | uagguaguuucauguuguugg | 21 | 70 |
| | sapiens | | | | |
| hsa-mir-197 | Homo | mir-197 | uucaccaccuucuccacccagc | 22 | 75 |
| | sapiens | | | | |
| hsa-mir-198 | Homo | mir-198 | gguccagaggggagauagg | 19 | 62 |
| | sapiens | | | | |
| hsa-mir-199a-2 | Homo | mir-199a-2 | cccaguguucagacuaccuguuc | 23 | 110 |
| | sapiens | | | | |
| hsa-mir-199b | Homo | mir-199b | cccaguguuuagacuaucuguuc | 23 | 110 |
| | sapiens | | | | |
| hsa-mir-199a-1 | Homo | mir-199a-1 | cccaguguucagacuaccuguuc | 23 | 110 |
| | sapiens | | | | |
| hsa-mir-199-s | Homo | mir-199-s | cccaguguucagacuaccuguu | 22 | 71 |
| | sapiens | | | | |
| hsa-mir-200b | Homo | mir-200b | cucuaauacugccugguaaugaug | 24 | 95 |
| | sapiens | | | | |
| hsa-mir-203 | Homo | mir-203 | gugaaauguuuaggaccacuag | 22 | 110 |
| | sapiens | | | | |
| hsa-mir-204 | Homo | mir-204 | uucccuuugucauccuaugccu | 22 | 110 |
| | sapiens | | | | |
| hsa-mir-205 | Homo | mir-205 | uccuucauuccaccggagucug | 22 | 110 |
| | sapiens | | | | |
| hsa-mir-208 | Homo | mir-208 | auaagacgagcaaaaagcuugu | 22 | 71 |
| | sapiens | | | | |
| hsa-mir-210 | Homo | mir-210 | cugugcgugugacagcggcug | 21 | 110 |
| | sapiens | | | | |
| hsa-mir-211 | Homo | mir-211 | uucccuuugucauccuucgccu | 22 | 110 |
| | sapiens | | | | |
| hsa-mir-212 | Homo | mir-212 | uaacagucuccagucacggcc | 21 | 110 |
| | sapiens | | | | |
| hsa-mir-213 | Homo | mir-213 | aacauucauugcugucgguggguu | 24 | 110 |
| | sapiens | | | | |
| hsa-mir-214 | Homo | mir-214 | acagcaggcacagacaggcag | 21 | 110 |
| | sapiens | | | | |
| hsa-mir-215 | Homo | mir-215 | augaccuaugaauugacagac | 21 | 110 |
| | sapiens | | | | |
| hsa-mir-216 | Homo | mir-216 | uaaucucagcuggcaacugug | 21 | 110 |
| | sapiens | | | | |
| hsa-mir-217 | Homo | mir-217 | uacugcaucaggaacugauuggau | 24 | 110 |
| | sapiens | | | | |
| hsa-mir-218-1 | Homo | mir-218-1 | uugugcuugaucuaaccaugu | 21 | 110 |
| | sapiens | | | | |
| hsa-mir-218-2 | Homo | mir-218-2 | uugugcuugaucuaaccaugu | 21 | 110 |
| | sapiens | | | | |
| hsa-mir-219 | Homo | mir-219 | ugauuguccaaacgcaauucu | 21 | 110 |
| | sapiens | | | | |
| hsa-mir-220 | Homo | mir-220 | ccacaccguaucugacacuuu | 21 | 110 |
| | sapiens | | | | |
| hsa-mir-221 | Homo | mir-221 | agcuacauugucugcuggguuuc | 23 | 110 |
| | sapiens | | | | |
| hsa-mir-222 | Homo | mir-222 | agcuacaucuggcuacugggucuc | 24 | 110 |
| | sapiens | | | | |
| hsa-mir-223 | Homo | mir-223 | ugucaguuugucaaauacccc | 21 | 110 |
| | sapiens | | | | |
| hsa-mir-224 | Homo | mir-224 | caagucacuagugguuccguuua | 23 | 81 |
| | sapiens | | | | |

In one embodiment, the mRNA is any of the mRNA listed in Table 1. In a preferred embodiment, the mRNA is abundant in the cell. In other embodiments, the mRNA is a let-7 mRNA, an miR124a mRNA, or miR166 mRNA. Other mRNA's for use in the present invention include miR9, miR124 and miR125 with tissue specific activity in the brain; miR143 with tissue specific activity in adipocytes (e.g., in adipocyte differentiation); miR1, miR133a, miR133b, miR1d, miR206d and miR296 with tissue specific activity in muscle; or, alternatively, miR192, miR194, miR215, miR216 and miR204 with tissue specific activity in the kidney. mRNA's for use in the present invention are well known in the art (see Griffiths-Jones S. “The microRNA Registry”, NAR (2004) 32, Database Issue, D109-D111 or through online searching at the Sanger Institute website, both of which are hereby incorporated herein by reference).

The mRNA recruiting moiety should be of sufficient size to effectively recruit the desired mRNA. The length of the recruiting moiety will vary greatly depending, in part, on the length of the mRNA and the degree of complementarity between the miRNA and the recruiting moiety. Generally, miRNAs are between about 17 to about 23 nucleotides in length. Accordingly, in various embodiments of the present invention, the miRNA recruiting moiety is less than about 25, 24, 23, 22, 21, 20, 19, 18, 17, 16, 15, 14, 13, 12, 11, 10, 9, 8, 7, 6, 5, 4, 3 or 2 nucleotides in length. In one embodiment, the recruiting moiety is about 13 to about 21 nucleotides in length. In another embodiment, the recruiting moiety is about 13, 14, 15 or 16 to 21 nucleotides in length. In a particular embodiment, the recruiting moiety is about 13, 14 or 15 nucleotides in length.

According to the invention, the linking moiety refers to a domain, portion or region of the RNA-silencing agent which covalently joins or links the miRNA targeting moiety and the miRNA recruiting moiety. The linking moiety merely tethers the targeting moiety and the recruiting moiety. Accordingly, the linking moiety may be a discrete entity as known in the art, including, but not limited to, a carbon chain, a nucleotide sequence, polyethylene glycol (PEG) or a cholesterol. Alternatively, the linking moiety may be a simple phosphorus-containing moiety, such as a phosphodiester linkage, a phosphorothioate, or a methylphosphonates. In a particular embodiment, the linking moiety is a phosphodiester bond. Moreover, the linking moiety may be modified as necessary (as described below) to optimize the stability of the RNA-silencing agent.

In one embodiment, the linking moiety is a nucleotide sequence. The linking moiety may be of any length suitable both to allow the binding of the moieties to their respective target miRNA and miRNA, and to promote the repression of the target miRNA by the miRNA. In one embodiment, the linking moiety is less than about 50, 30, 25, 20, 19, 18, 17, 16, 15, 14, 13, 12, 11, 10, 9, 8, 7, 6, 5, 4, 3, or 2 nucleotides in length. In a particular embodiment, the linking moiety is about 5 to about 10 nucleotides in length.

The silencing agent, and each of the miRNA targeting moiety, the miRNA recruiting moiety and the linking moiety should be designed as necessary so as to promote effective translational repression. Factors to be considered when designing the agent and the respective domains include, but are not limited to, enhancing the ability of the agent to recruit both the miRNA and the miRNA, in addition to enchancing the overall stability and cellular uptake of the agent.

A. Sequence Complementarity

The complementarity of the miRNA targeting moiety and the miRNA recruiting moiety should be designed to promote binding of miRNA and miRNA, respectively. The targeting moiety should include a sequence of sufficient size and of sufficient degree of complementarity to the target miRNA so as to effectively and selectively bind the target miRNA. In one embodiment of the invention, the RNA-silencing agent contains a targeting moiety with sufficient complementarity to a plurality of sites on a target miRNA sequence (e.g., about 10, 5, 4, 3, or 2 sites). In another embodiment, the RNA-silencing agent contains a plurality of targeting moieties, each with sufficient complementarity to one or more sites on the target miRNA sequence. In a particular embodiment, at least two of the targeting moieties may have sufficient complementarity to the same site on the target miRNA sequence. Alternatively, the RNA-silencing agent contains a targeting moiety with complementarity to one site on a target miRNA sequence.

Similarly, the recruiting moiety should include a region of both sufficient size and of sufficient degree of complementarity to the desired miRNA so as to effectively and selectively bind the desired miRNA. In one embodiment, the RNA-silencing agent contains a recruiting moiety with sufficient complementarity to a plurality of miRNAs. In another embodiment, the RNA-silencing agent contains a plurality of recruiting moieties, each with sufficient complementarity to at least one miRNA. In a particular embodiment, at least two of the recruiting moieties may have sufficient complementarity to the same miRNA. Alternatively, the RNA-silencing agent contains a recruiting moiety with sufficient complementarity to one miRNA.

Designing sequences in terms of size and complementarity to optimize binding to target sequences is well known in the art. The recruiting moiety and/or the targeting moiety may have 100% sequence identity to the miRNA and the miRNA, respectively. However, 100% identity is not required. Greater than 90% sequence identity, e.g., 91%, 92%, 93%, 94%, 95%, 96%, 97%, 98%, 99% or even 100% sequence identity, between the targeting moiety and the miRNA and/or the recruiting moiety and the miRNA is preferred. Generally, however, the sequence identity should be that which is sufficient to promote selective binding of the moieties to their respective targets. The invention, thus, has the advantage of being able to tolerate sequence variations that might be expected due to genetic mutation, strain polymorphism, or evolutionary divergence.

B. Modifications

In another embodiment of the invention, the RNA-silencing agent, any of the respective moities and, in particular, the linking moiety, are modified such that the in vivo activity of the agent is improved without compromising the agent's RNA silencing activity. The modifications can, in part, serve to enhance stability of the agent (e.g., to prevent degradation), to promote cellular uptake, to enhance the target efficiency, to improve efficacy in binding (e.g., to the targets), to improve patient tolerance to the agent, and/or to reduce toxicity.

RNA-silencing agents of the invention can be modified at the 5′ end, 3′ end, 5′ and 3′ end, and/or at internal residues, or any combination thereof. In one embodiment, the RNA-silencing agent of the invention comprises one or more (e.g., about 1, 2, 3, 4, 5, 6, 7, 8, 9, 10, or more) end modifications. Modification may be at the 5′ end or the 3′ end.

In certain embodiments, the internal residues of the RNA-silencing agents (e.g., the linking moiety) are modified. As defined herein, an “internal” nucleotide is one occurring at any position other than the 5′ end or 3′ end of a nucleic acid molecule, polynucleotide or oligonucleotide. An internal nucleotide can be within a single-stranded molecule or within either strand of a duplex or double-stranded molecule. In one embodiment, the RNA-silencing agent (preferably the linking moiety within an RNA-silencing agent) is modified by the substitution of at least one internal nucleotide. In another embodiment, the RNA-silencing agent is modified by the substitution of at least 2, 3, 4, 5, 6, 7, 8, 9, 10, 11, 12, 13, 14, 15, 16, 17, 18, 19, 20, 21, 22, 23, 24, 25 or more internal nucleotides. In another embodiment, the RNA-silencing agent (preferably the linking moiety within an RNA-silencing agent) is modified by the substitution of at least 5%, 10%, 15%, 20%, 25%, 30%, 35%, 40%, 45%, 50%, 55%, 60%, 65%, 70%, 75%, 80%, 85%, 90%, 95% or more of the internal nucleotides. In yet another embodiment, the linking moiety within the RNA-silencing agent is modified by the substitution of all of the internal nucleotides.

Internal modifications can be, for example, sugar modifications, nucleobase modifications, backbone modifications. Alternatively, the modified RNA-silencing agent can contain mismatches or bulges. In one embodiment, the RNA-silencing agent of the invention comprises one or more (e.g., about 1, 2, 3, 4, 5, 6, 7, 8, 9, 10, or more) backbone-modified nucleotides (i.e., modifications to the phosphate sugar backbone). For example, the phosphodiester linkages of natural RNA may be modified to include at least one of a nitrogen or sulfur heteroatom. In preferred backbone-modified ribonucleotides the phosphoester group connecting to adjacent ribonucleotides is replaced by a modified group, e.g., of phosphothioate group.

In another embodiment, the RNA-silencing agent of the invention includes sugar-modified nucleotides. The 2′ moiety can be, but is not limited to, H, OR, R, halo, SH, SR, NH₂, NHR, NR₂ or ON, wherein R is C₁-C₆ alkyl, alkenyl or alkynyl and halo is F, Cl, Br or I. In particular embodiments, the modifications are 2′-fluoro, 2′-amino and/or 2′-thio modifications. Particularly preferred modifications include 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, and/or 5-amino-allyl-uridine. In a particular embodiment, the 2′-fluoro ribonucleotides are every uridine and cytidine. Additional exemplary modifications include 5-bromo-uridine, 5-iodo-uridine, 5-methyl-cytidine, ribo-thymidine, 2-aminopurine, 2′-amino-butyryl-pyrene-uridine, 5-fluoro-cytidine, and 5-fluoro-uridine. 2′-deoxy-nucleotides and 2′-Ome nucleotides can also be used within modified RNA-silencing agents moities of the instant invention. Additional modified residues include, deoxy-abasic, inosine, N3-methyl-uridine, N6, N6-dimethyl-adenosine, pseudouridine, purine ribonucleoside and ribavirin. In a particularly preferred embodiment, the 2′ moiety is a methyl group such that the linking moiety is a 2′-O-methyl oligonucleotide.

In another embodiment, the RNA-silencing agent (e.g., the linking moiety) of the invention comprises one or more (e.g., about 1, 2, 3, 4, 5, 6, 7, 8, 9, 10, or more) nucleobase-modified nucleotides (i.e., the nucleotides contain at least one non-naturally occurring nucleobase instead of a naturally occurring nucleobase). Bases may be modified to block the activity of adenosine deaminase. Exemplary modified nucleobases include, but are not limited to, uridine and/or cytidine modified at the 5-position (e.g., 5-(2-amino)propyl uridine, 5-fluoro-cytidine, 5-fluoro-uridine, 5-bromo-uridine, 5-iodo-uridine, and 5-methyl-cytidine), adenosine and/or guanosines modified at the 8 position (e.g., 8-bromo guanosine), deaza nucleotides (e.g., 7-deaza-adenosine), and O- and N-alkylated nucleotides (e.g., N6-methyl adenosine). Nucleobase-modified nucleotides for use in the present invention also include, but are not limited to, ribo-thymidine, 2-aminopurine, 2,6-diaminopurine, 4-thio-uridine, and 5-amino-allyl-uridine and the like.

In another embodiment, the RNA-silencing agent of the invention comprises a sequence wherein at least a portion (e.g., the miRNA targeting moiety or the miRNA recruiting moiety) contains one or more (e.g., about 1, 2, 3, 4, 5, 6, 7, 8, 9, 10, or more) mismatches with the respective target (e.g., miRNA or miRNA). In another embodiment (e.g., where at least a portion of the RNA-silencing agent is double stranded, the RNA-silencing agent of the invention comprises a bulge, for example, one or more (e.g., about 1, 2, 3, 4, 5, 6, 7, 8, 9, 10, or more) unpaired bases in one of the strands.

In another embodiment, the RNA-silencing agent of the invention comprises any combination of two or more (e.g., about 2, 3, 4, 5, 6, 7, 8, 9, 10, or more) modifications as described herein. For example, the RNA-silencing agent can comprise a combination of two sugar-modified nucleotides, wherein the sugar-modified nucleotides are 2′-fluoro modified ribonucleotides (e.g., 2′-fluoro uridine or 2′-fluoro cytidine) and 2′-deoxy ribonucleotides (e.g., 2′-deoxy adenosine or 2′-deoxy guanosine).

According to the invention, the RNA-silencing agent should be modified as necessary, in part, to improve stability, to prevent degradation in vivo (e.g., by cellular nucleases), to improve cellular uptake, to enhance target efficiency, to improve efficacy in binding (e.g., to the targets), to improve patient tolerance to the agent, and/or to reduce toxicity.

In one embodiment, the RNA-silencing agent has an miRNA targeting moiety or portion of about 25 to about 50 nucleotides in length. The targeting moiety or portion is on the 5′ end of the silencing agent. Adjacent the targeting moiety or portion is the linking moiety or portion. The linking moiety or portion is about 5 to about 10 nucleotides in length and has at least one modified nucleotide (e.g., a 2′-O-methyl nucleotide or a phosphorothiate nucleotide). On the 3′ end of the agent, adjacent the linker, is a miRNA recruiting moiety or portion which is about 5 to about 25 nucleotides in length. Optionally, the RNA-silencing agent may have additional modifications in the flanking portions or moieties of the agent.

In one embodiment, the RNA-silencing agent has an miRNA targeting moiety or portion of about 25 to about 50 nucleotides in length. The targeting moiety or portion is on the 3′ end of the silencing agent. Adjacent the targeting moiety or portion is the linking moiety or portion. The linking moiety or portion is about 5 to about 10 nucleotides in length and has at least one modified nucleotide (e.g., a 2′-O-methyl nucleotide or a phosphorothiate nucleotide). On the 5′ end of the agent, adjacent the linker, is a miRNA recruiting moiety or portion which is about 5 to about 25 nucleotides in length. Optionally, the RNA-silencing agent may have additional modifications in the flanking portions or moieties of the agent.

Methods of Treatment

The present invention further provides for methods for treating a subject (e.g., a human) having or at risk for a disease or disorder. The disease may be characterized or caused by the overexpression or overactivity of a cellular protein, or alternatively, may be caused by the expression or activity of a mutant protein. Accordingly, administration of an RNA-silencing agent that has an miRNA targeting moiety capable of binding the miRNA encoding the overexpressed, overactive or mutant protein, can serve to repress the translation of the target miRNA. The disease may be genetic (e.g., a neurodegenerative disease such as Huntington's Disease which is caused by expression of mutant huntingtin protein) or non-genetic. In another embodiment, the disease is characterized or caused by a gain-of-function mutant protein (e.g., SOD1).

In certain embodiments, the RNA silencing agents of the invention can be used to identify and/or validate potential targets for therapeutic interventions against diseases or disorders, for example, cancer, viral infections, chronic pain and other diseases described herein. The RNA silencing agents of the invention can be used for target identification and/or validation animal models or, alternatively, in appropriate cell culture models. Animal models include, but are not limited to, mammalian models, for example, rodent models (e.g., mouse or rat models), as well as non-mammalian biological systems, for example, Drosophila systems, C. elegans and the like. Cell culture models feature, for example human primary cells, human cell lines, rodent cell lines, Drosophila cells, C. elegans cells, etc. Target validation methods of the invention involve, for example, administering a RNA silencing agent of the invention to a cell or organism comprising a potential therapeutic target and determining the effect of the silencing agent on one or more biological processes or activities associated with the target. In one embodiment, a target is potentially involved a process, such as processes including but not limited to, cell growth, proliferation, apoptosis, morphology, angiogenesis, differentiation, migration, viral multiplication, drug resistance, signal transduction, cell cycle regulation, morphogenesis, senescence, mitosis, meiosis, temperature sensitivity, chemical sensitivity, nerve cell growth, bacterial cell growth, plant cell growth, stress tolerance, biosynthesis of cellular factors or metabolites, viral resistance, bacterial resistance, or resistance to infection by a pathogen and others. A RNA silencing agent specific for the target is administered to an appropriate cell or animal model under conditions sufficient for silencing of the target and the effect of the silencing agent on the process is determined. In another embodiment, a target is potentially involved in a disease or disorder or other pathophisiological condition and the RNA silencing agent specific for the target is administered to an appropriate cell or animal model under conditions sufficient for silencing of the target and the effect of the silencing agent on the disease or disorder or other pathophisiological condition is determined. The effect of the silencing agent can be determined as a direct effect on expression or activity of the target or the expression or activity of a downstream molecule or process effected or regulated by said target. The effect of the silencing agent can be determined as an effects on a process regulated by or associated with said target. The effect of the silencing agent can be determined as an effect on a biological characteristic or phenotype associated with said target. In appropriate animal models, for example, in animal models of disease or disorder, the effect of the silencing agent can be determined as an improvement, reversal, or attenuation is the disease or disorder or one or more symptoms or biological features of the disease or disorder.

The compositions and methods of the present invention can serve to validate particular targets for further study, for example, ultimately for the treatment of a disease or disorder. For example, using the techniques of the present invention, the effects of the repression of particular genes on cellular function may be analyzed.

The present invention provides for both prophylactic and therapeutic methods of treating a subject at risk of (or susceptible to) a disorder or having a disorder associated with aberrant or unwanted target gene expression or activity. In achieving a therapeutic or prophylactic effect, the compositions and methods of the present invention have the added advantage of inducing translational repression only in those cells that express the endogenous miRNA for which the RNA silencing agent is designed to recruit. Accordingly, the RNA silencing agent may be freely administered with the knowledge that undesirable translational repression will not occur in non-targeted cells, thereby providing a tissue specificity for the compositions and methods of the present invention. The risk of undesirable translational repression is further minimized by the teachings of the present invention in that RNA silencing agents can be designed to target multiple sequences in a gene. Indeed, as the number of gene target sites are increased, the probability that the RNA silencing agent will induce translational repression in an undesirable gene is similarly reduced.

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the RNA-silencing agents of the present invention according to that individual's drug response genotype. Pharmacogenomics allows a clinician or physician to target prophylactic or therapeutic treatments to patients who will most benefit from the treatment and to avoid treatment of patients who will experience toxic drug-related side effects.

A. Prophylactic Methods

In one aspect, the invention provides a method for preventing in a subject, a disease or condition associated with an aberrant or unwanted target gene expression or activity, by administering to the subject a therapeutic agent (e.g., an RNA-silencing agent). Subjects at risk for a disease which is caused or contributed to by aberrant or unwanted target gene expression or activity can be identified by, for example, any or a combination of diagnostic or prognostic assays as described herein. Administration of a prophylactic agent can occur prior to the manifestation of symptoms characteristic of the target gene aberrancy, such that a disease or disorder is prevented or, alternatively, delayed in its progression. Depending on the type of target gene aberrancy, for example, a target gene, target gene agonist or target gene antagonist agent can be used for treating the subject. The appropriate agent can be determined based on screening assays described herein.

B. Therapeutic Methods

Another aspect of the invention pertains to methods of modulating target gene expression, protein expression or activity for therapeutic purposes. Accordingly, in an exemplary embodiment, the modulatory method of the invention involves contacting a cell capable of expressing a target gene with a therapeutic agent (e.g., an RNA-silencing agent) that is specific for the target gene or protein (e.g., is specific for the miRNA encoded by said gene or specifying the amino acid sequence of said protein) such that expression or one or more of the activities of target protein is modulated. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a target gene polypeptide or nucleic acid molecule. Inhibition of target gene activity is desirable in situations in which the target gene is abnormally unregulated and/or in which decreased target gene activity is likely to have a beneficial effect.

C. Animal Models

Therapeutic agents can be tested in an appropriate animal model. For example, an RNA-silencing agent as described herein can be used in an animal model to determine the efficacy, toxicity, or side effects of treatment with said agent. Alternatively, a therapeutic agent can be used in an animal model to determine the mechanism of action of such an agent. For example, an agent can be used in an animal model to determine the efficacy, toxicity, or side effects of treatment with such an agent. Alternatively, an agent can be used in an animal model to determine the mechanism of action of such an agent.

D. Disease Indications

In one embodiment, the present invention provides methods for the treatment of diseases associated with gain-of-function mutations using the RNA-silencing agents disclosed herein. The term “gain-of-function mutation” as used herein, refers to any mutation in a gene in which the protein encoded by said gene (i.e., the mutant protein) acquires a function not normally associated with the protein (i.e., the wild type protein) causes or contributes to a disease or disorder. The gain-of-function mutation can be a deletion, addition, or substitution of a nucleotide or nucleotides in the gene which gives rise to the change in the function of the encoded protein. In one embodiment, the gain-of-function mutation changes the function of the mutant protein or causes interactions with other proteins. In another embodiment, the gain-of-function mutation causes a decrease in or removal of normal wild-type protein, for example, by interaction of the altered, mutant protein with said normal, wild-type protein.

“Allele specific inhibition of expression” refers to the ability to significantly inhibit expression of one allele of a gene over another, e.g., when both alleles are present in the same cell. For example, the alleles can differ by one, two, three or more nucleotides. In some cases, one allele is associated with disease causation, e.g., a disease correlated to a dominant gain-of-function mutation.

Diseases caused by dominant, gain-of-function gene mutations develop in heterozygotes bearing one mutant and one wild type copy of the gene. Some of the best-known diseases of this class are common neurodegenerative diseases, including Alzheimer's disease, Huntington's disease (associated with mutant huntingtin), Parkinson's disease (associated with mutant parkin), amyotrophic lateral sclerosis (ALS; “Lou Gehrig's disease”) (associated with mutant superoxide dismutase-1 (SOD1)) (Taylor et al., 2002) and autosomal dominant disorders. In these diseases, the exact pathways whereby the mutant proteins cause cell degeneration are not clear, but the origin of the cellular toxicity is known to be the mutant protein.

Mutations in SOD1 cause motor neuron degeneration that leads to ALS, because the mutant protein has acquired some toxic property (Cleveland et al., 2001). Neither the nature of this toxic property nor the downstream pathway that leads to the eventual motor neuron degeneration is understood. In mice, only expression of the mutant SOD1, but not elimination of SOD1 by gene knockout, causes ALS. Nonetheless, the gene knockout mice develop numerous abnormalities including reduced fertility (Matzuk et al., 1990), motor axonopathy (Shefner et al., 1999), age-associated loss of cochlear hair cells (McFadden et al., 2001) and neuromuscular junction synapses (Flood et al., 1999), and enhanced susceptibility to a variety of noxious assaults, such as excitotoxicity, ischemia, neurotoxins and irradiation, on the CNS and other systems (Matz et al., 2000; Kondo et al., 1997; Kawase et al., 1999; Behndig et al., 2001). Given the toxicity of the mutant and the functional importance of the wild-type protein, the ideal therapy for this disease would selectively block the expression of the mutant protein while retaining expression of the wild type.

Huntington's Disease

In one embodiment, the present invention provides methods for the treatment of Huntington's Disease (HD) using the RNA-silencing agents disclosed herein. Huntington's disease complies with the central dogma of genetics: a mutant gene serves as a template for production of a mutant miRNA; the mutant miRNA then directs synthesis of a mutant protein (Aronin et al, Neuron; DiFiglia and Aronin, Science; others). Mutant huntingtin (protein) probably accumulates in selective neurons in the striatum and cortex, disrupts as yet determined cellular activities, and causes neuronal dysfunction and death (Aronin, Philos. Transactions; Laforet and Aronin, J. Neurosci., others). Because a single copy of a mutant gene suffices to cause Huntington's disease, the most parsimonious treatment would render the mutant gene ineffective. Theoretical approaches might include stopping gene transcription of mutant huntingtin, destroying mutant miRNA, and blocking translation. Each has the same outcome—loss of mutant huntingtin.

The disease gene linked to Huntington's disease is termed Huntington or (htt). The huntingtin locus is large, spanning 180 kb and consisting of 67 exons. The huntingtin gene is widely expressed and is required for normal development. It is expressed as 2 alternatively polyadenylated forms displaying different relative abundance in various fetal and adult tissues. The larger transcript is approximately 13.7 kb and is expressed predominantly in adult and fetal brain whereas the smaller transcript of approximately 10.3 kb is more widely expressed. The two transcripts differ with respect to their 3′ untranslated regions (Lin et al., 1993). Both messages are predicted to encode a 348 kilodalton protein containing 3144 amino acids. The genetic defect leading to Huntington's disease is believed to confer a new property on the miRNA or alter the function of the protein.

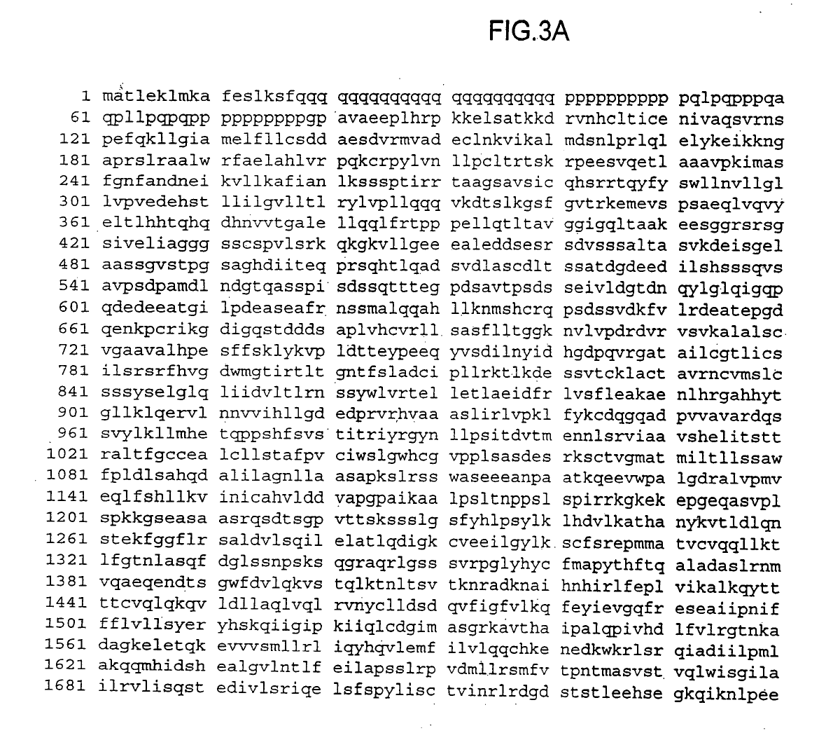

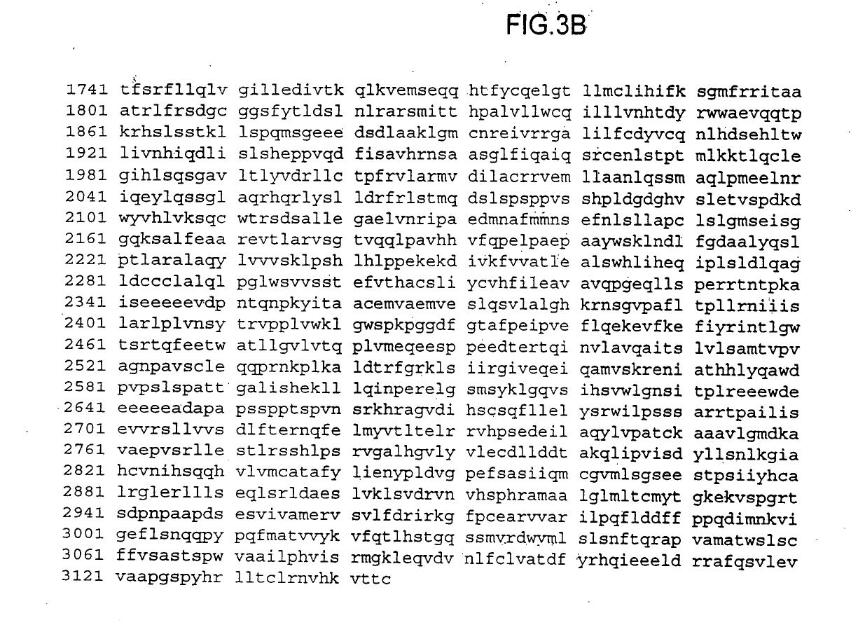

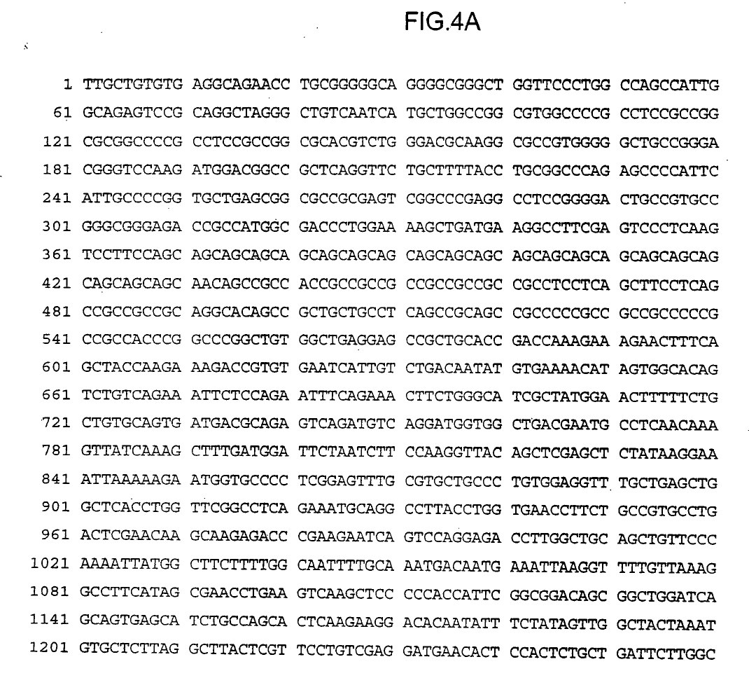

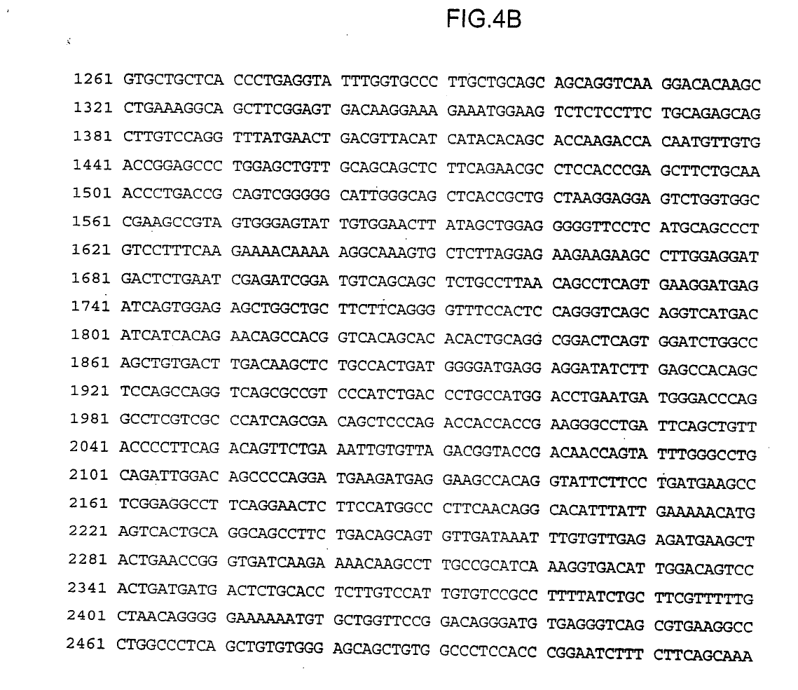

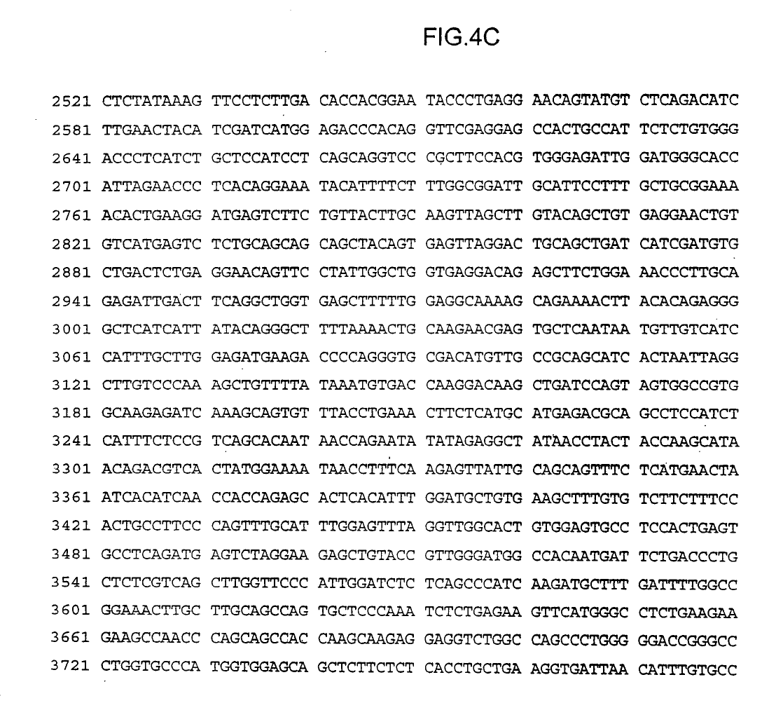

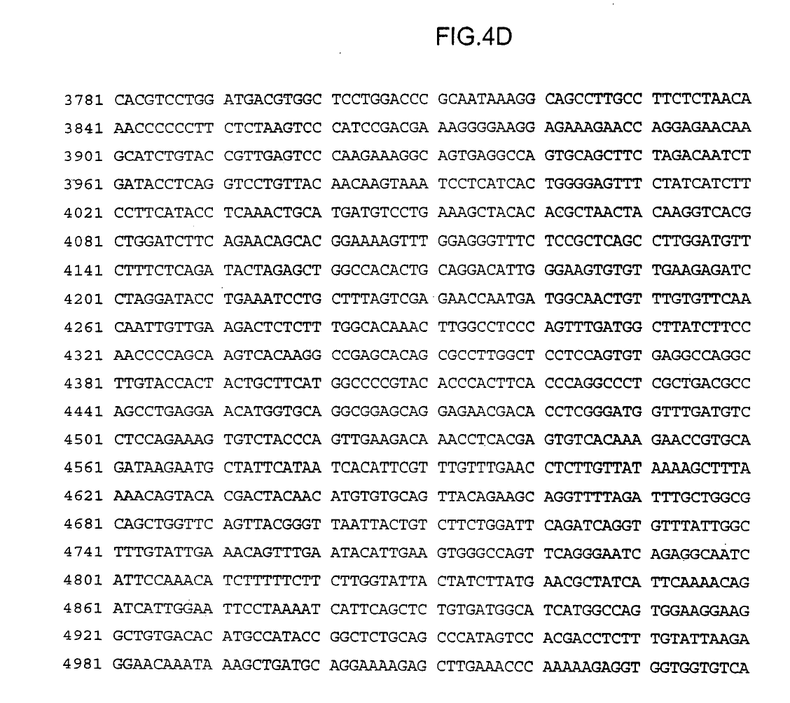

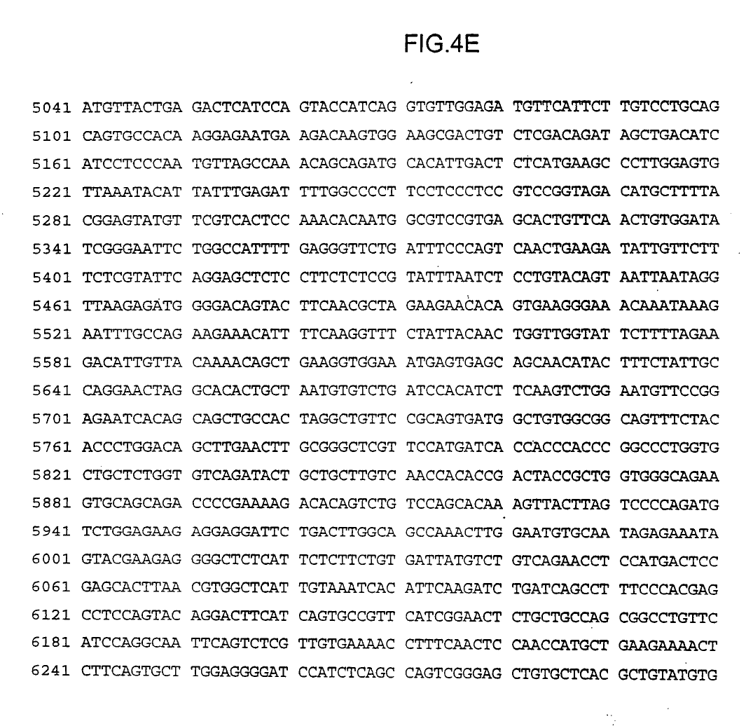

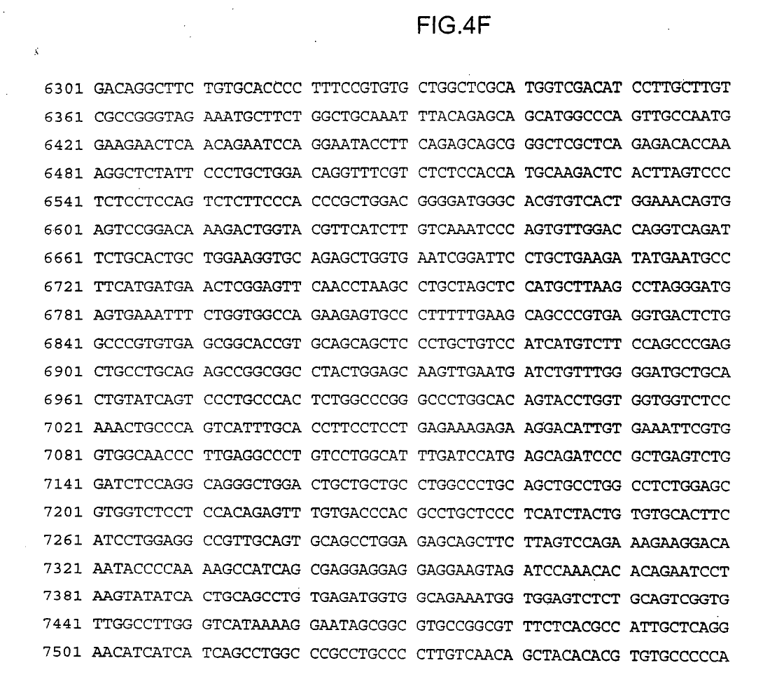

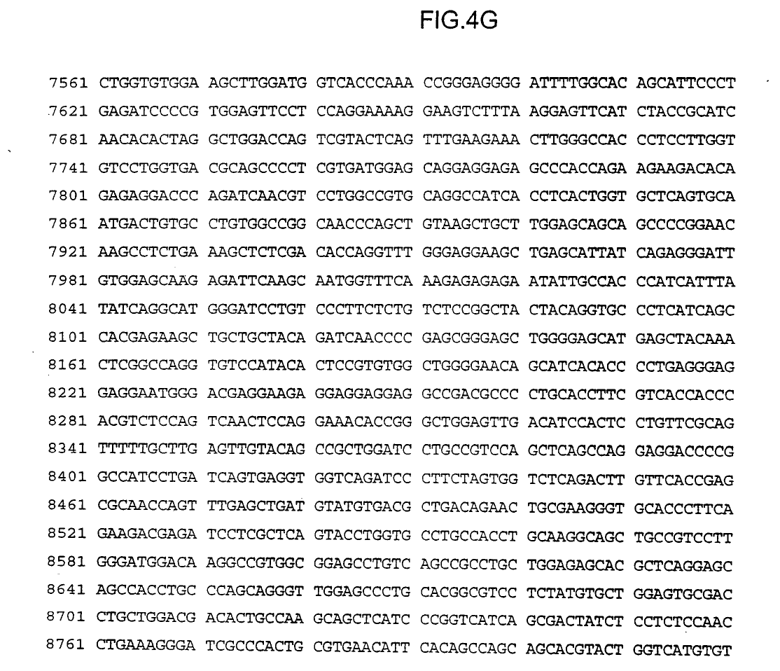

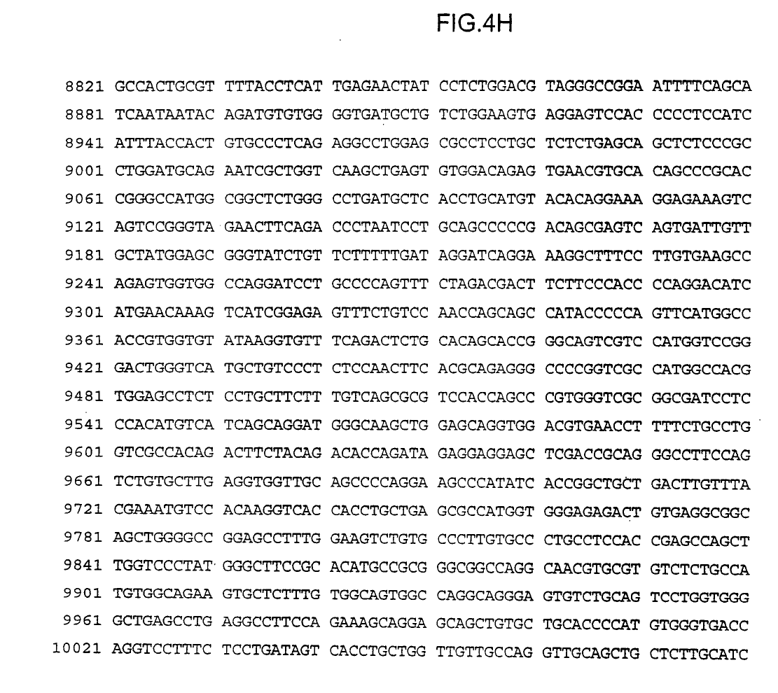

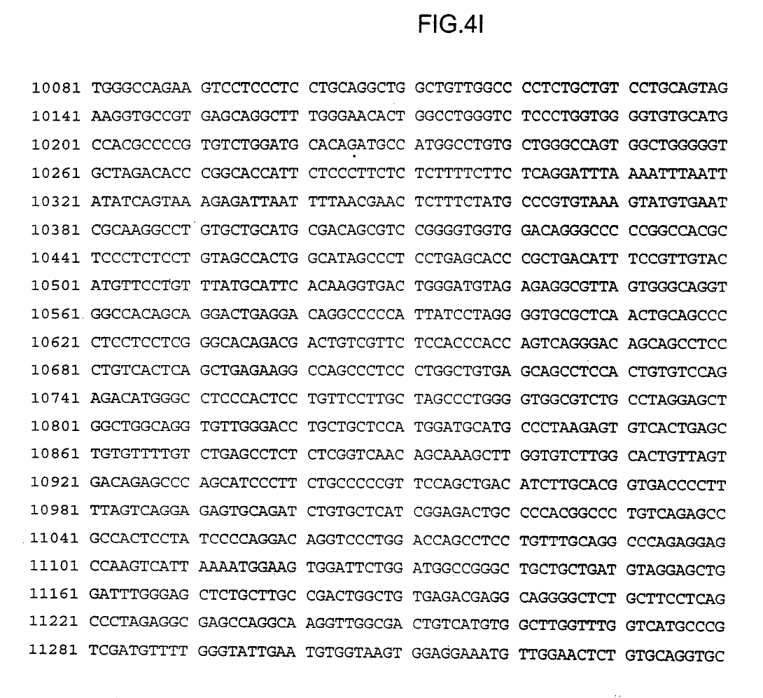

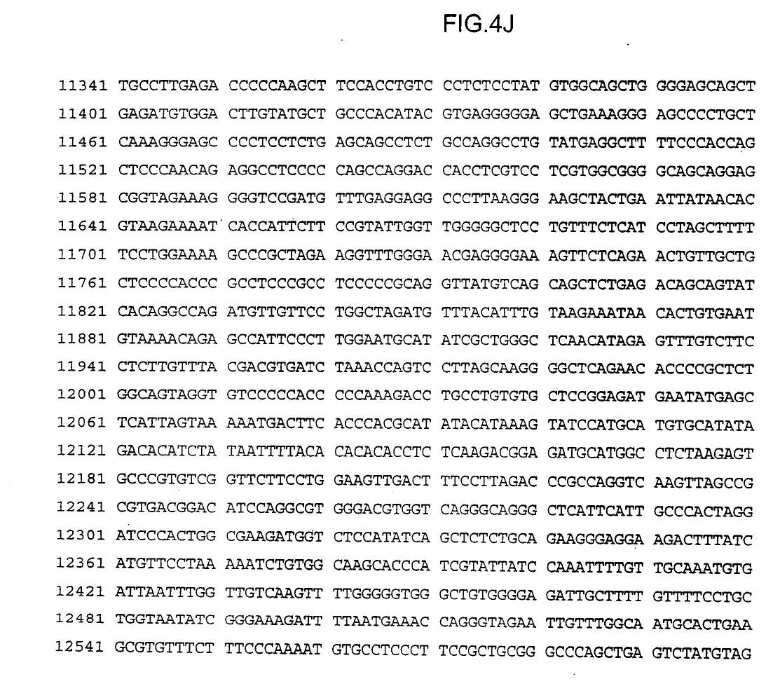

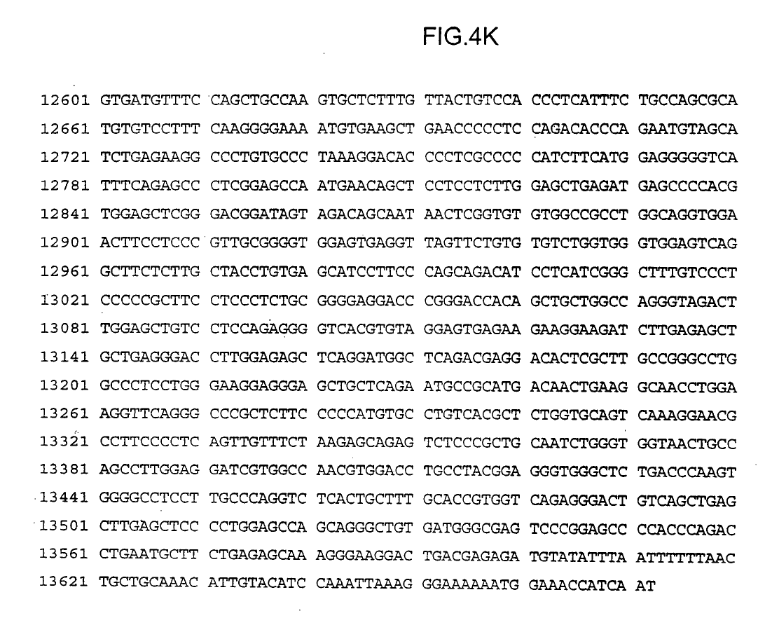

The amino acid sequence of the human huntingtin protein is set forth in FIG. 3 (SEQ ID NO:1). The nucleotide sequence of the human huntingtin gene (cDNA) is set forth in FIG. 4 (SEQ ID NO:2). The coding region consists of nucleotides 316 to 9750 of SEQ ID NO:2.

Other Indications

In other embodiments, the compositions of the invention can act as novel therapeutic agents for controlling one or more of cellular proliferative and/or differentiative disorders, disorders associated with bone metabolism, immune disorders, hematopoietic disorders, cardiovascular disorders, liver disorders, viral diseases, pain or metabolic disorders.

For example, in various embodiments, the miRNA targeting moiety can target an miRNA encoding a protein (e.g., a mutant protein) selected from the group consisting of matrix metalloproteinase 1, matrix metalloproteinase 2, matrix metalloproteinase 9, metalloelastase, CD36 receptor, tenascin-C, secreted protein acidic and rich in cysteine (SPARC), and androgen receptor gene. Without wishing to be bound to any particular theory, it is believed that these proteins may be involved in cellular proliferative disorders.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer,” “hyperproliferative,” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol/Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

In general, the compositions of the invention are designed to target genes associated with particular disorders. Examples of such genes associated with proliferative disorders that can be targeted include activated ras, p53, BRCA-1, and BRCA-2.

The compositions of the invention can be used to treat a variety of immune disorders, in particular those associated with overexpression of a gene or expression of a mutant gene. Examples of hematopoietic disorders or diseases include, but are not limited to, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjogren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy such as, atopic allergy.

Examples of disorders involving the heart or “cardiovascular disorder” include, but are not limited to, a disease, disorder, or state involving the cardiovascular system, e.g., the heart, the blood vessels, and/or the blood. A cardiovascular disorder can be caused by an imbalance in arterial pressure, a malfunction of the heart, or an occlusion of a blood vessel, e.g., by a thrombus. Examples of such disorders include hypertension, atherosclerosis, coronary artery spasm, congestive heart failure, coronary artery disease, valvular disease, arrhythmias, and cardiomyopathies.

Disorders which may be treated by methods described herein include, but are not limited to, disorders associated with an accumulation in the liver of fibrous tissue, such as that resulting from an imbalance between production and degradation of the extracellular matrix accompanied by the collapse and condensation of preexisting fibers.

Additionally, molecules of the invention can be used to treat viral diseases, including but not limited to hepatitis B, hepatitis C, herpes simplex virus (HSV), HIV-AIDS, poliovirus, and smallpox virus. Molecules of the invention are engineered as described herein to target expressed sequences of a virus, thus ameliorating viral activity and replication. The molecules can be used in the treatment and/or diagnosis of viral infected tissue. Also, such molecules can be used in the treatment of virus-associated carcinoma, such as hepatocellular cancer.

Pharmaceutical Compositions

The invention pertains to uses of the above-described RNA-silencing agents for therapeutic treatments as described infra. Accordingly, the RNA-silencing agents of the present invention can be incorporated into pharmaceutical compositions suitable for administration. Such compositions typically comprise the RNA-silencing agent or other modulatory compound and a pharmaceutically acceptable carrier. As used herein the language “pharmaceutically acceptable carrier” is intended to include any and all solvents, dispersion media, coatings, antibacterial and antifingal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In various embodiments, the pharmaceutical composition of the present invention includes an RNA-silencing agent and an agent suitable for delivery to a subject. Alternatively, the invention includes an RNA-silencing agent conjugated to an agent suitable for delivery to a subject. Suitable delivery agents include, but are not limited to, proteinaceous agents (e.g., peptides), hydrophobic agents or lipid-based agents.

A pharmaceutical composition of the invention is formulated to be compatible with its intended route of administration. Examples of routes of administration include parenteral, e.g., intravenous, intradermal, subcutaneous, intraperitoneal, intramuscular, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. Although compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography.

A therapeutically effective amount of a composition containing a compound of the invention (e.g., an RNA-silencing agent) (i.e., an effective dosage) is an amount that inhibits expression of the polypeptide encoded by the target gene by at least 30 percent. Higher percentages of inhibition, e.g., 45, 50, 75, 85, 90 percent or higher may be preferred in certain embodiments. Exemplary doses include milligram or microgram amounts of the molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. The compositions can be administered one time per week for between about 1 to 10 weeks, e.g., between 2 to 8 weeks, or between about 3 to 7 weeks, or for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments.

It is furthermore understood that appropriate doses of a composition depend upon the potency of composition with respect to the expression or activity to be modulated. When one or more of these molecules is to be administered to an animal (e.g., a human) to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The pharmaceutical compositions can be included in a container, pack, or dispenser together with instructions for administration This invention is further illustrated by the following examples which should not be construed as limiting.

### EXEMPLIFICATION

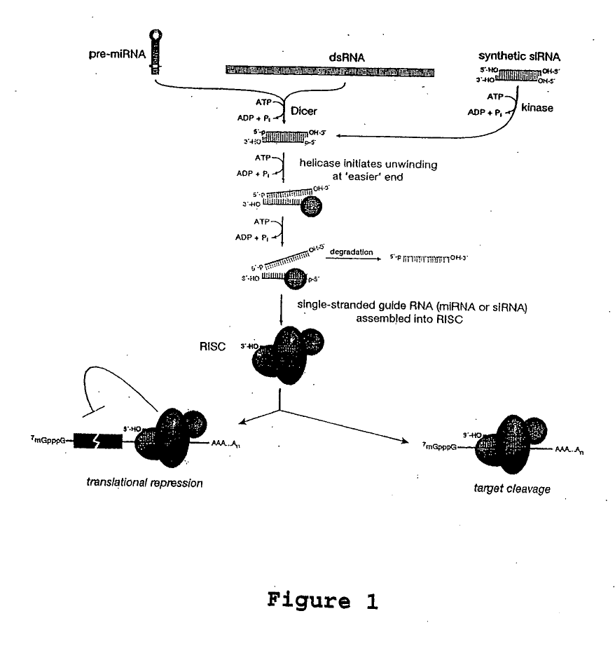

RNA silencing encompasses two main types of small effecter molecules, siRNA and miRNA (FIG. 1; Lau et al. (2001) Science 294:858-862; Lee et al. (2001) Science 294:862-864). siRNA is an RNA duplex; it operates by destruction of miRNA. miRNA is a single stranded RNA molecule that prevents protein production by blocking translation. miRNA has been shown to associate with RISC in translational repression (Doench et al. (2003) Genes Dev 17:438-442; Hutvanger et al. (2001) Science 293:834-838; Khvorova et al. (2003) Trends Biotechnol 21:74-81; Schwarz et al. (2003) Cell 115:199-208; Zeng et al. (2003) Proc Natl Acad Sci USA 100:9779-9784). Furthermore, recent studies demonstrate that miRNAs are present in mammalian cells and, particularly, in neurons (Krichevsky et al. (2003) RNA 9:1274-1281; Lagos-Quintana et al. (2002) Current Biol 12:735-739; Logos-Quintana et al. (2001) Science 294:853-858; Lim et al. (2003) Science 299:1540). However, the roles of miRNAs remain unsolved in mammalian cells. miRNA may have different, perhaps complementary effects compared to siRNA in reducing mutant proteins in cells. The following examples describe inducing translational repression in cells and neurons by targeting miRNA and RISC, for example, to huntingtin miRNA.

### Example 1

### Probing miRNA as an Effector in RNA Silencing

miRNA binds to miRNA sites through nucleotide complementarity. Like siRNA, miRNA forms complexes with RISC proteins. The mechanism by which miRNA blocks translation is unknown. Mammalian cells have ˜250 distinct miRNAs (Lagos-Quintana et al. (2002); Logos-Quintana et al. (2001); Lim et al. (2003)). In the instant example, a miRNA in abundance is recruited to the htt miRNA using a 2′-O-methyl oligonucleotide complementary to both the miRNA and the miRNA target. 2′-O-methyl oligonucleotides have been shown to be irreversible, stoichiometric inhibitors of both siRNA and miRNA function (Hutvagner et al. (2004) PLOS Biology, in press). The method recruits the miRNA-programmed RISC to the miRNA and block synthesis of mutant huntingtin protein.

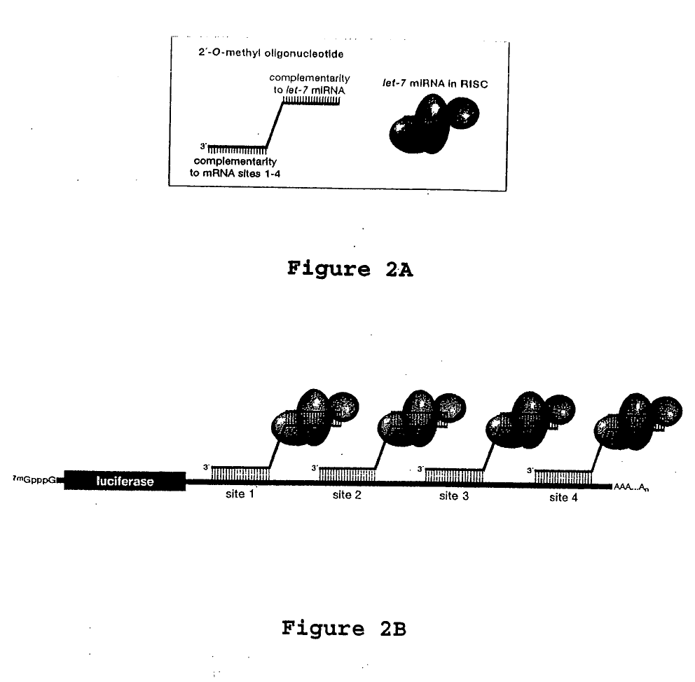

FIG. 2 depicts interactions between the designed 2′-O-methyl oligonucleotide and an endogenous miRNA. FIG. 2 further depicts the general design of an embodiment of the 2′-O-methyl oligonucleotide appropriate for the present example. The 3′ end of the oligonucleotide is designed to bind to miRNA. The 5′ end of the oligonucleotide is complementary to the sequence of an endogenous miRNA, in this case let-7. The identification of many different endogenous miRNAs in mammalian cells and in neurons allows flexibility in designing and testing agents of the invention for miRNA-dependent effectiveness of translational repression. The diagram shows four sites of oligonucleotide complementarity to miRNA. Four sites are shown to be more effective than one to three sites for the luciferase reporter, and are proposed to be similarly effective for endogenous miRNA translational repression. The gray spheres depict RISC proteins associated with the endogenous miRNA.

For the present example, 2′-O-methyl oligonucleotides are synthesized with two functional domains: an oligonucleotide region against a 3′-UTR sequence in a luciferase reporter miRNA and a domain complementary to let-7 miRNA, an abundant and potent miRNA in HeLa cells (Hutvagner et al. (2002) Science 297:2056-2060). The luciferase miRNA is engineered to have four sites for oligonucleotide complementation, so that the proximal 5′ part of the oligonucleotide binds to these four identical 21 nucleotide ‘sites’ in series. Because the oligonucleotide contains a sequence fully complementary to the let-7 miRNA, the oligonucleotide-endogenous miRNA complex is proposed to attract RISC, prior to attachment to the Renilla reniformis luciferase miRNA. Subsequently, reagents are transfected into HeLa cells with Lipofectamine 2000 and, after 24 hours, cells are harvested to test for luciferase activity by standard assays.

Analysis. Controls include (1) transfection of luciferase cDNA without miRNA oligonucleotide to show basal luciferase reporter activity and (2) transfection of luciferase cDNA plus oligonucleotide without let-7 miRNA, having no effect on luciferase activity. In all experiments, an internal firefly luciferase control is included for normalization. The 2′-O-methyl oligonucleotide lacks modifications necessary to attract RISC (5′ phosphate, 3′-OH, nucleotide overhangs). siRNA duplexes that bind to the four 3′ UTR sites with imperfect complementarity and that repress translation of the reporter by an miRNA-like mechanism are also utilized (Dykxhoorn et al. (2003) Nature Reviews, Molecular Cell Biology 4:457-467). Differences in luciferase reporter activities are compared with ANOVA and Bonferroni correction, to establish significance (p<0.05). At least six separate tests are carried out.

### Example 2

### miRNA Translation Repression of Mutant Huntingtin Protein

In the instant example, miRNA recruitment to effect translation repression of huntingtin is tested. Initial test paradigms, under controlled conditions, are established before testing for huntingtin miRNA. Since huntingtin miRNA is susceptible to siRNA-directed RNAi in HeLa cells, the first study uses 2′-O-methyl oligonucleotide against huntingtin miRNA in HeLa cells. Three tests are applied. First, 2′-O-methyl oligonucleotides directed against huntingtin miRNA sequences with let-7 miRNA-complementary extensions are constructed as shown in FIG. 2. Huntingtin miRNA sites (six in series) are inserted into a luciferase reporter (FIG. 2). Translational repression is measured by luciferase activity in a luminometer. Next, the oligonucleotide is transfected into HeLa cells and huntingtin protein is measured on Western blots. Huntingtin is quantified on LAS3000 (Fuji, Stamford, Conn.). Controls include transfection of miRNA against luciferase (absent in these cells) and huntingtin siRNA, to compare effectiveness of RNAi. The 2′-O-methyl oligonucleotide effect on translational repression in neuronally derived cells also is tested. The above experimental design is repeated in X-57 cells, which are transfected with the GFP mutant huntingtin cDNA. Counts of GFP cells are used to estimate transfection efficiency. With use of an NSE promoter, about 50% transfection effiency of GFP has been demonstrated. Huntingtin protein is measured on Western blots, as an estimate of translational repression. Repression of huntingtin measured in immunoblots is then compared to endogenous a-tubulin on LAS3000 (Fuji). The same controls and statistical analysis as used in Example 1 are applied here. Tests are repeated at least 6 times for analysis. Statistical analysis includes ANOVA and Bonferroni corrections.

Blocking translation by the designed 2′-O-methyl oligonucleotide indicates that an miRNA and, by implication, RISC need only be proximate to the target miRNA. miRNA arrays are expected to disclose high abundance miRNAs in brain and such studies are under active investigation (Krichevsky et al. (2003) RNA 9:1274-1281). Results from these studies will enable testing of several endogenous miRNA constructs. This information will provide candidate participants in RNA silencing to the central nervous system. Furthermore, since RNA silencing by miRNA invokes translational repression and siRNA destroys miRNA, miRNA can provide additional flexibility in formulating RNA silencing strategies. For example, a gentle knock down of mutant huntingtin might suffice to reduce HD pathogenesis without excessive huntingtin loss that could harm cells.

Let-7 is a well-recognized miRNA, known to be active in mammalian cells (Hutvanger et al. (2002)). Other miRNAs have been identified in mouse tissue, although biological activity is not yet secured (Lagos-Quintana et al. (2001)). Especially abundant in mouse brain, in cortex, is miR124a (Lagos-Quintana et al. (2002)). miR124a activity is examined as a substitute for let-7. Other single strand oligonucleotides should be considered as an alternative to siRNA. Locked nucleic acids are modified nucleotides that resist nuclease activities (highly stable) and possess single nucleotide discrimination for miRNA (Braasch et al. (2003) Biochemistry 42:7967-7975, Petersen et al. (2003) Trends Biotechnol 21:74-81). These molecules have 2′-O,4′-C-ethylene-bridged nucleic acids, with possible modifications such as 2′-deoxy-2″-fluorouridine. An experimental alternative, is to examine locked nucleic acids to improve stability and single nucleotide selectivity in cells and in vivo. miRNA oligonucleotide in X-57 neurons can also be utilized.

### Example 3

### Exploring the Requirements for siRNA Translational Repression

In both plants and animals, siRNAs are perfectly complementary to their targets, directing cleavage of the RNA target at the middle of the binding site. In contrast, animal miRNAs usually act as sequence specific translational repressors. About one percent of animal genes encode miRNAs, many of which are evolutionally conserved, and they regulate diverse cellular functions, including developmental timing, cell proliferation, cell death, and fat metabolism. Both endogenous miRNAs and exogenous siRNA's can direct the destruction of an miRNA at any single binding site to which they are sufficiently complementary. In contrast, miRNAs and siRNAs that are insufficiently complementary to support cleavage of the RNA target can nonetheless direct translational repression if the target contains multiple, partially complementary RNA binding sites in the 3′ untranslated region. The following experiments utilized RNA modifications to coax siRNAs that are perfectly complementary to their targets to elicit translational repression. The following experiments used modified nucleic acid tethers to recruit endogenous miRNA or transfected siRNA to an unrelated target RNA and to repress its expression.

Generally, reporter plasmids pGL-2, expressing Photinus pyralis Luciferase, and pRL-TK, expressing Renilla reniformis Luciferase were co-transfected with or without 2′-O-methyl oligonucleotide or siRNA in HeLa cells in 24 well plate format. Amount of reporter vector DNA per well was 0.025 μg of pRL-TK plasmid and contained the appropriate target sites for RNA silencing agent (2′-O-methyl oligonucleotide tethers) and 0.05 μg of pGL-2 plasmid. Concentration of RNA silencing agent and siRNA transfected per well ranged as indicated below. Cells were transfected in 600 μL of Opti-MEM (Gibco) and incubated for 24 hours. 48 hours post transfection cells were washed in 500 μL PBS and 100 μL of Passive Lysis Buffer (Promega) was added to each well. 24 well plates were incubated at room temperature for 20 min. Plates were then subjected to two freeze thaw cycles. An aliquot of 10 μL of HeLa lysate was analyzed for luciferase activity according to the Promega Dual Luciferase Assay in a mediators PhL luminometer. Renilla luciferase activity level was divided by the corresponding Firefly luciferase activity level to normalize Renilla levels between transfections. Where appropriate, base line Renilla luciferase was determined from the sample that received control oligonucleotide sense to Renilla ORF or GFP siRNA and was considered 100% Renilla luciferase expression.

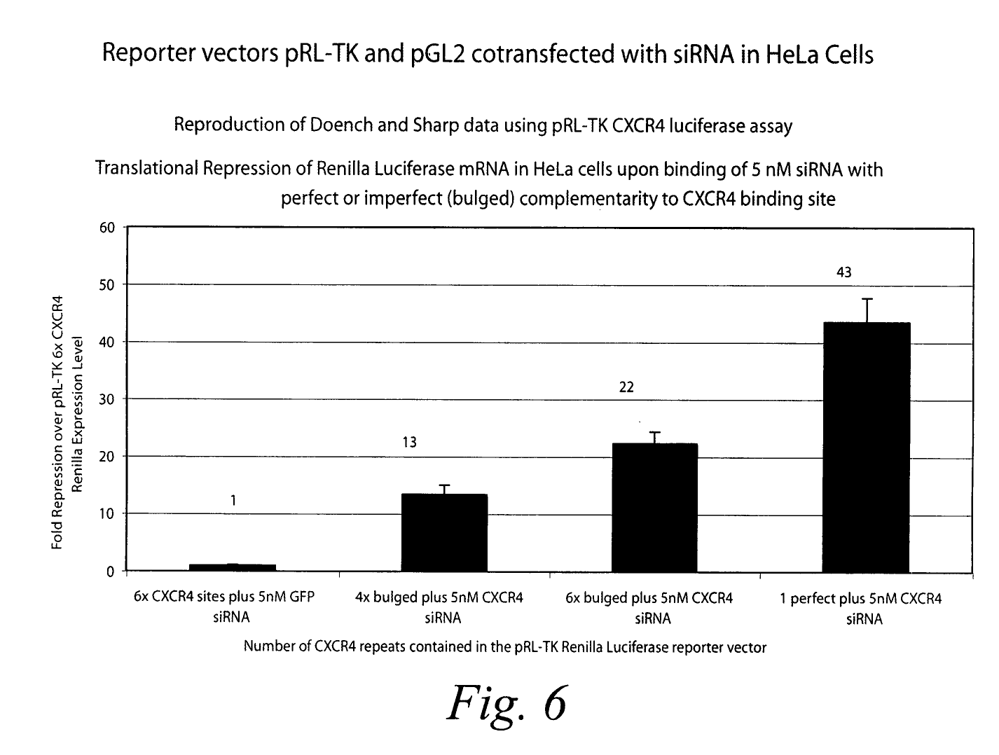

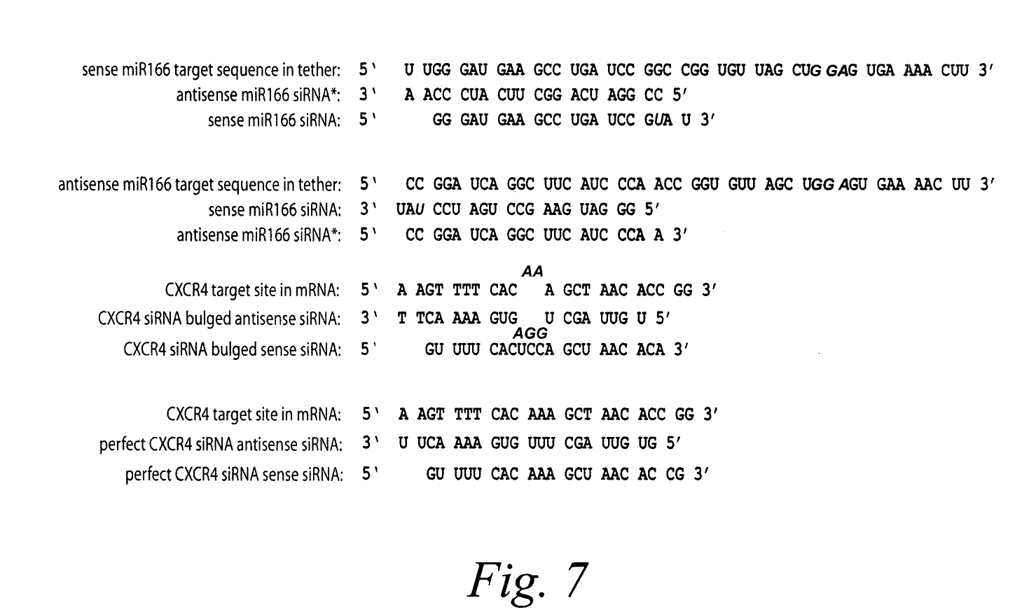

FIG. 6 depicts translational repression of Renilla luciferase miRNA in HeLa cells upon binding of 5 nM siRNA with perfect or imperfect (bulged) complementarity to CXCR4 binding site. The HeLa cells were cotransfected with reporter vectors pRL-TK and pGL2 and siRNA. The sequences utilized are as shown in FIG. 7. Luciferase expression was measured using pRL-TK CXCR4 luciferase assay as described in Doench et al. (2003). As shown therein, “6×CXCR4 sites plus 5 nM GFP siRNA” represents the control, and appropriately little or no gene silencing was demonstrated. “4× bulged plus 5 nM CXCR4 siRNA” represents the transfection of the HeLa cells with bulged siRNA (as shown in FIG. 7) and targeted to four binding sites. “6× bulged plus 5 nM CXCR4 siRNA” represents the transfection of the HeLa cells with bulged siRNA (as shown in FIG. 7) and targeted to six binding sites. Lastly, “1 perfect plus 5 nm CXCR4 siRNA” represents transfection with a perfectly complementary siRNA, believed to induce cleavage of the RNA target. Appropriately, increasing the number of binding sites increased translational repression.

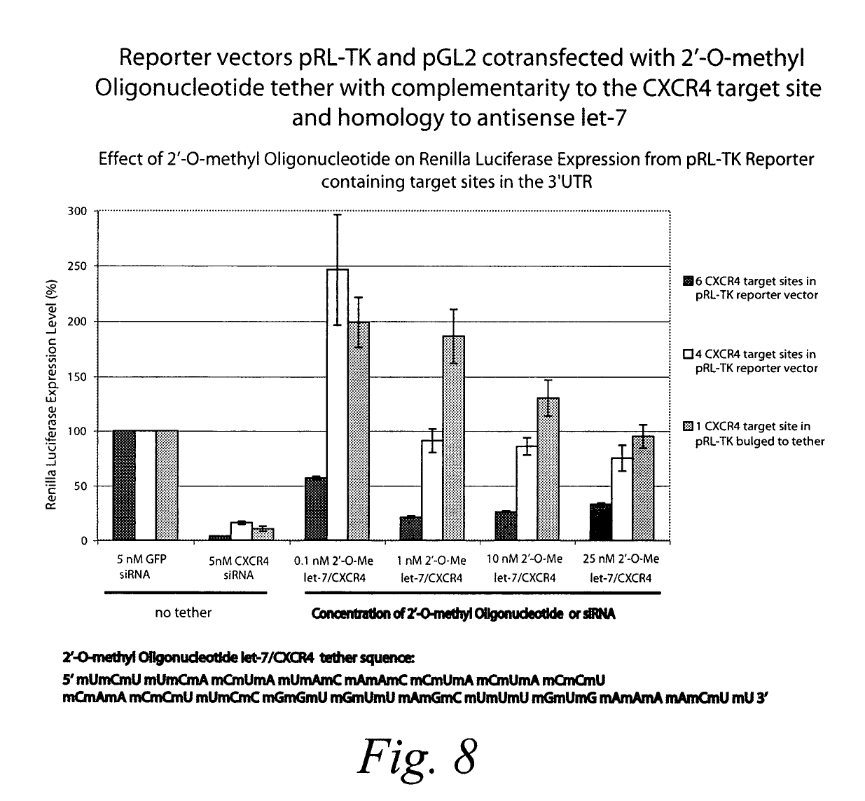

FIG. 8 depicts the effect of 2′-O-methyl oligonucleotide RNA silencing agents on Renilla luciferase expression. Specifically, reporter vectors pRL-TK and pGL2 were contransfected with 2′-O-methyl oligonucleotide RNA silencing agents with complementarity to the CXCR4 target sites and with homology to antisense miRNA let 7. As indicated in FIG. 8, two controls were run, one in which HeLa cells were transfected with 5 nM GFP siRNA and one in which cells were transfected with 5 nM CXCR4 siRNA to induce cleavage of the target gene. In the experimental runs, 2′-O-methyl oligonucleotide RNA silencing agents (as shown in FIG. 8) were administered at varying concentrations. Each of the control and experimental runs were designed to target either 1, 4 or 6 target sites in the pRL-TK reporter vector. Appropriately, targeting more sites induced greater translational repression. Indeed, as shown in FIG. 8, 0.1 nM of the RNA silencing agent was sufficient to induce substantial translational repression when designed to target six sites.

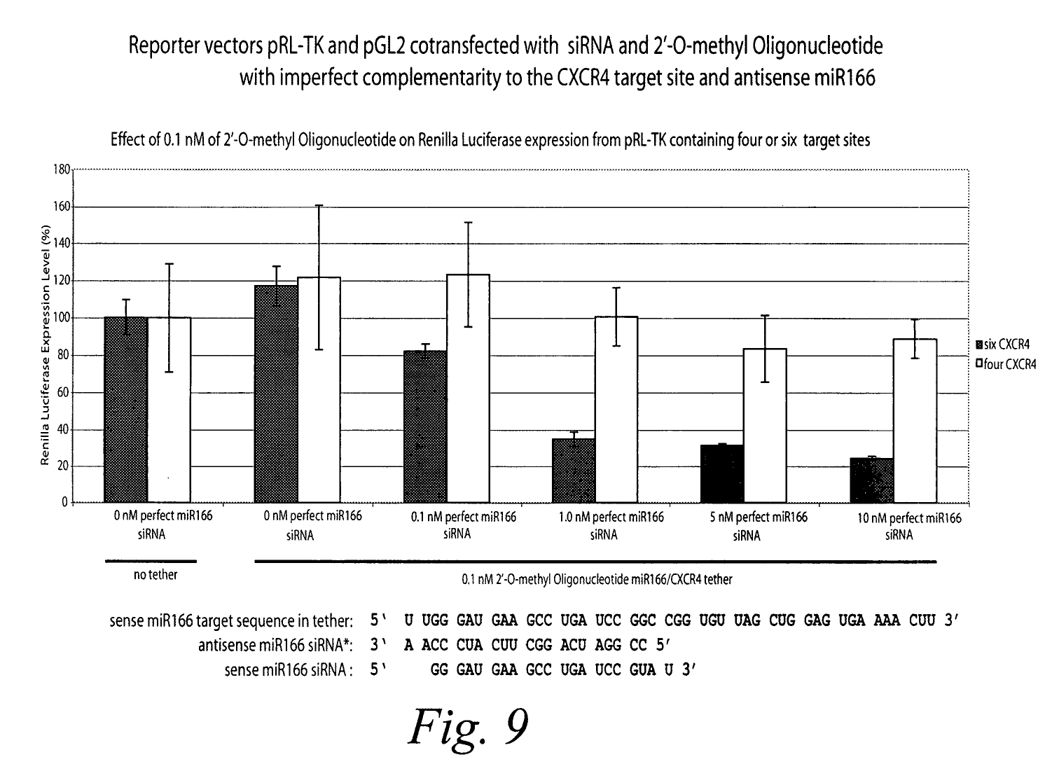

FIG. 9 similarly depicts the effect of 2′-O-methyl oligonucleotide RNA silencing agents on Renilla luciferase expression. Specifically, reporter vectors pRL-TK and pGL2 were contransfected with siRNA and 2′-O-methyl oligonucleotide RNA silencing agents with imperfect complementarity to the CXCR4 target sites and antisense miR166. As indicated in FIG. 9, a control was run in which HeLa cells were transfected with the luciferase system but no siRNA or 2′-O-methyl oligonucleotide miR166/CXCR4 tethers. In the experimental runs, perfect miR166 siRNA at varying concentrations was cotransfected with 0.1 nM 2′-O-methyl oligonucleotide miR166/CXCR4 tethers (as shown in FIG. 9). Each of the control and experimental runs were designed to target either 4 or 6 CXCR4 target sites. As shown therein, targeting more sites had a greater effect on luciferase expression.

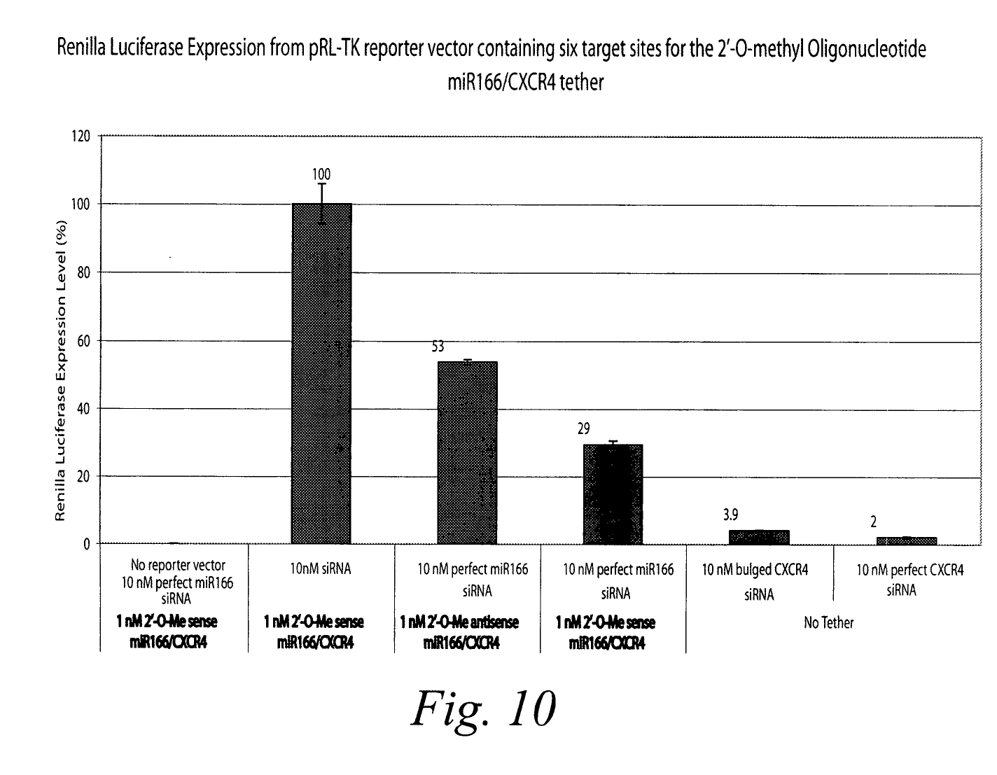

FIG. 10 shows Renilla luciferase expression from HeLa cells transfected with pRL-TK reporter vectors containing six target sites for the 2′-O-methyl oligonucleotide miR166/CXCR4 tether. Controls included transfection of either 10 nM bulged CXCR4 siRNA or 10 nM perfect CXCR4 siRNA, with no 2′-O-methyl oligonucleotide tether. The low levels of luciferase expression are believed to be a result of induced RNA target cleavage. In experimental runs, HeLa cells were transfected with or without perfect miR166 siRNA and with either 1 nm of the 2′-O-methyl oligonucleotide sense or antisense miR166/CXCR4 tether. The various sequences utilized are shown in FIG. 7. As shown in FIG. 10, the administration of 2′-O-methyl oligonucleotide sense miR166/CXCR4 along with the miR166 siRNA allowed for capture of the target sites of the gene and the appropriate miR166 to induce translational repression.

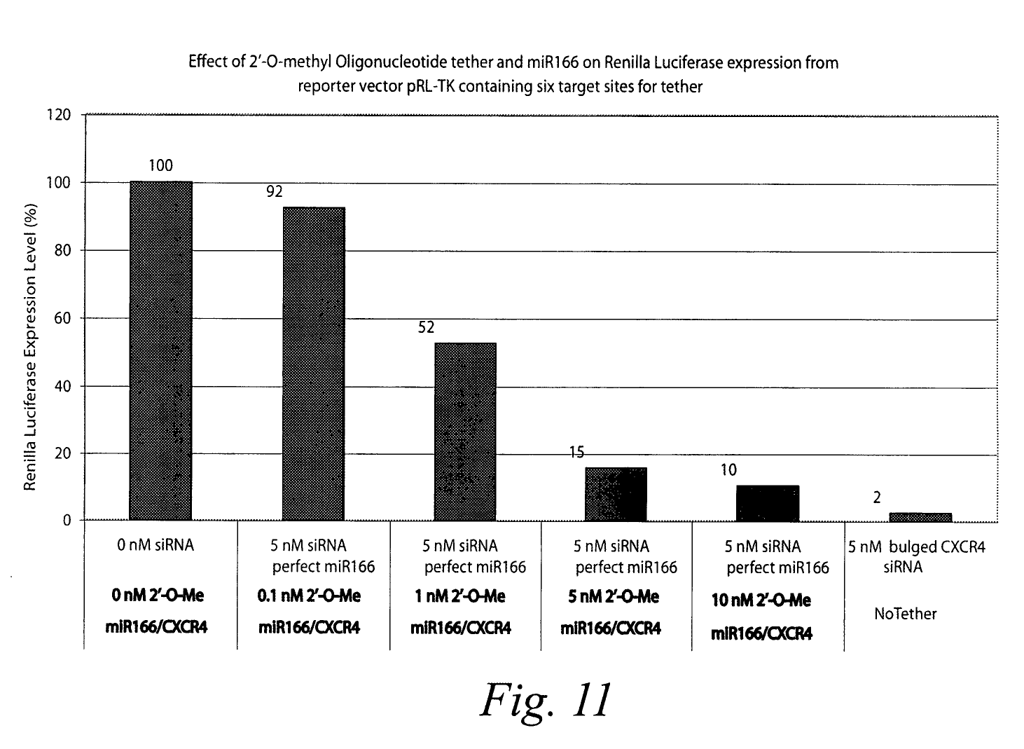

FIG. 11 shows the effect of 2′-O-methyl oligonucleotide tethers and miR166 on Renilla luciferase expression from reporter vector pRL-TK containing six target sites for the tether. In various runs, HeLa cells were transfected with 5 nM siRNA perfect miR166 along with varying concentrations of 2′-O-methyl oligonucleotide miR166/CXCR4 tethers. The various sequences utilized are shown in FIG. 7. Appropriately, increased concentrations of 2′-O-methyl oligonucleotide tether enhanced translational repression of luciferase.

### Example 4

### Effecting Gene Silencing of Target miRNA in Human Cells

The capacity for small RNAs to shut down specific gene activity is a result of nucleic acid base-pairing between target miRNA and effector small interfering RNA molecules that are tightly bound to the RNA induced silencing complex (RISC). A challenge to making successful small interfering RNA has been protecting the siRNA from nucleolytic degradation. That miRNAs exist in mammalian cells has made possible a new approach to gene silencing. A stable synthetic oligonucleotide has been created to recruit an endogenous miRNA to effect gene silencing of a specific target miRNA. The oligonucleotide tether has 2′-O-methyl substitutions, which confer resistance to degradation. The oligonucleotide has two regions of complementarity: one to the target miRNA and one to a miRNA. The miRNA let-7 is abundant in HeLa cells. The oligonucleotide tether binds the endogenous RNA-induced silencing complex through sequence complementarity to let-7 miRNA. A luciferase assay was used to measure luciferase activity in transiently transfected HeLa cells. 93% reduction in luciferase activity was achieved from an exogenous transcript. Without wishing to be bound to any particular theory, it is believed tha the mechanism by which the luciferase activity is decreased is translational repression of the transcript and not its degradation. Furthermore, it is believed that gene silencing is possible even though the RISC is not directly bound to the target miRNA but is recruited to the miRNA through the oligonucleotide tether. It is further believed that the oligonucleotide tether binds to the target miRNA with one region of complementarity and can effect gene silencing by recruiting the RISC proximal to the transcripts.

Results indicate that (1) it is possible to harness the function of an endogenous miRNA to effect gene silencing; (2) gene silencing does not require that the RISC bind directly to the target miRNA; and (3) it is possible to program the oligonucleotide tether to be active in target tissues by selecting which miRNA the tether will recruit.

Generally, the HeLa cells were transfected with the appropriate reporter plasmids as described in Example 3.

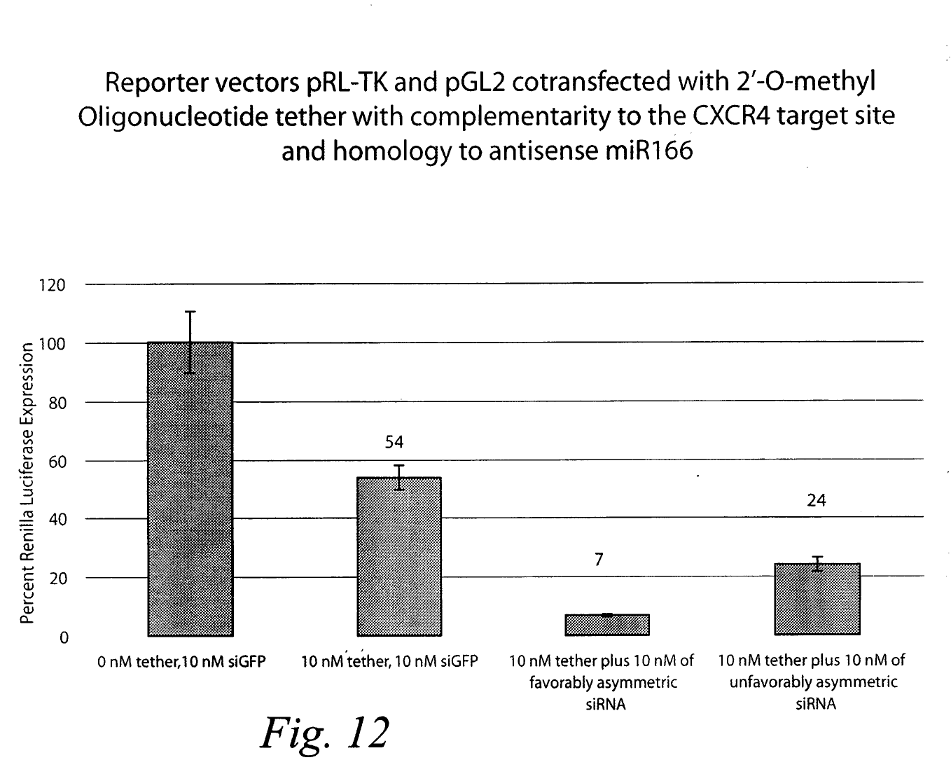

FIG. 12 depicts the percent Renilla luciferase expression in HeLa cells cotransfected with reporter vectors pRL-TK and pGL2 with 2′-O-methyl oligonucleotide tether with complementarity to the CXCR4 target site and with homology to antisense miR166. In various experimental runs, 10 nM of the oligonucleotide tether was cotransfected with either 10 nM of favorably asymmetric siRNA or 10 nM of unfavorably asymmetric siRNA. The favorably asymmetric siRNA was designed so as to desirably compel the antisense sequence of the siRNA into RISC and effect translational repression. By contrast the unfavorably asymmetric siRNA was designed so as to undesirably compel the sense sequence of the siRNA into RISC and hinder translational repression. The results as shown in FIG. 12 confirm the expected affects of the siRNA transcripts on luciferase expression.

### Example 5

### Examination of Effectiveness of RNA Silencing Agents Having Minimal miRNA Target Moiety Sequences and Minimal miRNA Recruiting Moiety Sequences

RNA silencing agent function was examined by a Luciferase Reporter Assay in HeLa cells transfected with Renilla luciferase encoding plasmid. The Renilla luciferase encoding plasmid had six target sites for binding the silencing agent, specifically, the miRNA targeting moiety, in the 3′ UTR of the gene. In the experimental culture of HeLa cells, the Renilla luciferase encoding plasmid, silencing agent and miRNA were transfected into cells with cationic lipid reagent. As one control, the HeLa cells did not naturally express the required endogenous miRNA. Instead, a plant miRNA, miR166, was transfected into HeLa cells in the experimental culture. Accordingly, the silencing agent was designed to recruit the miR166. This system allowed for assessment of any antisense effects of the silencing agent alone. In a control culture of HeLa cells, the Renilla luciferase encoding plasmid and the silencing agent were transfected into the HeLa cells. However, instead of the plant miRNA miR166, GFP siRNA was transfected into the HeLa cells.

The respective substrates for luciferin were added to the cell lysates. The activity of the luciferase, identifiable by its wavelength of its luminescence, was measured in each control and experimental sample.

Yet another control to which all experimental samples were compared was a culture that received a silencing agent that could not bind the Renilla miRNA or target sites because the miRNA targeting moiety contained the sense sequence instead of the complement of the target sequence.

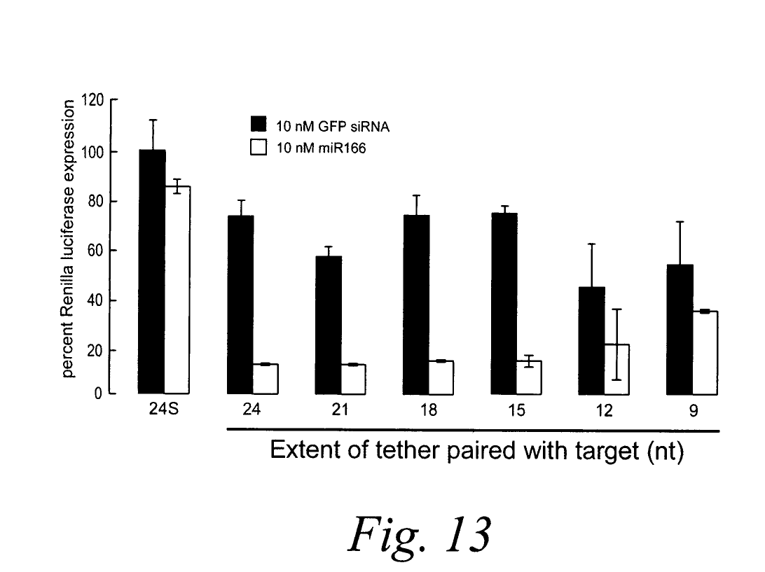

The initial analysis of oligonucleotide tether function focused on the determination of the minimal sequence needed to specifically bind the target miRNA, i.e. the miRNA targeting moiety. The 3′ end of the silencing agent was truncated by three nucleotides from 24 to 21, 18, 15, 12 and 9 nucleotides in length. Each silencing agent was tested for its ability to reduce Renilla luciferase activity in the Dual Luciferase Reporter Assay. The results of the truncations of the miRNA targeting moiety are shown in FIG. 13. As shown therein, optimal repression of luciferase expression relative to control was achieved with an miRNA targeting moiety of 15 nucleotides in length, although each truncated moiety was effective in repressing luciferase expression. (Note: “24 S” depicts the repression of luciferase expression where the miRNA targeting moiety is the sense sequence of the target miRNA.)

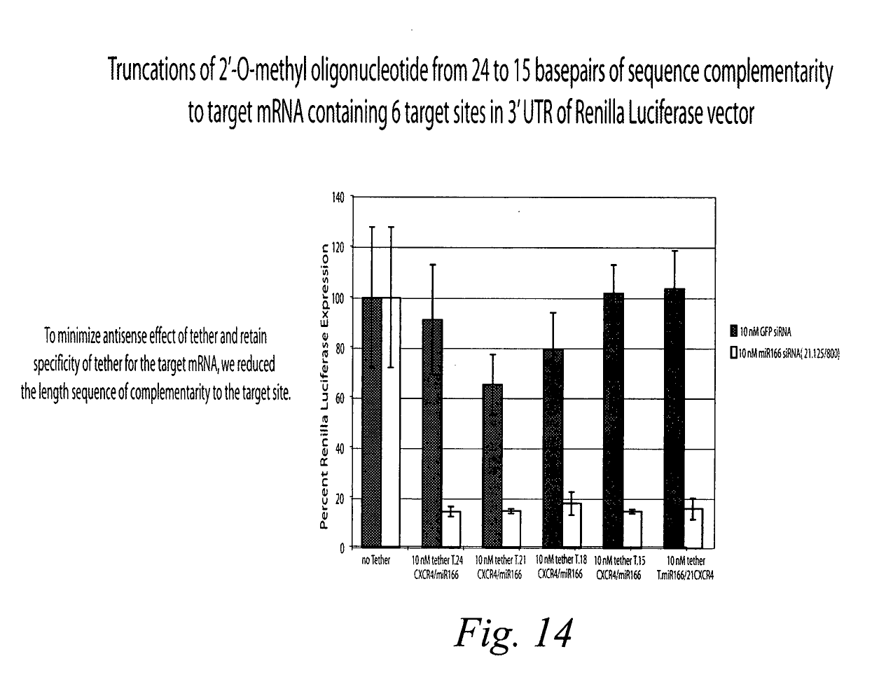

An additional analysis of truncation of the 3′ end of the 2′-O-methyl oligonucleotide tether, i.e., the miRNA targeting moiety is shown in FIG. 14. This analysis confirms that an miRNA targeting moiety of only 15 nucleotides in length is effective in repressing luciferase expression.

The 5′ end of the silencing agent required to recruit RISC, i.e., the miRNA recruiting moiety, was also truncated. Truncations of the original 21 nucleotide sequence of the miRNA recruiting moiety within the RNA silencing agent were made in two nucleotide increments to 19, 17, 15, and 13. The miRNA targeting moiety of the RNA silencing agents utilized in each of these experiments was the 15 nucleotide sequence identified above. Each RNA silencing agent was tested for its ability to reduce Renilla luciferase activity in the Dual Luciferase Reporter Assay. As above, the HeLa cells did not naturally express endogenous miRNA. Instead, the cells of the experimental culture were transfected with a plant miRNA, miR166, for which the miRNA recruiting moieties were designed to recruit. In the control culture of HeLa cells, GFP siRNA was transfected into the HeLa cells.

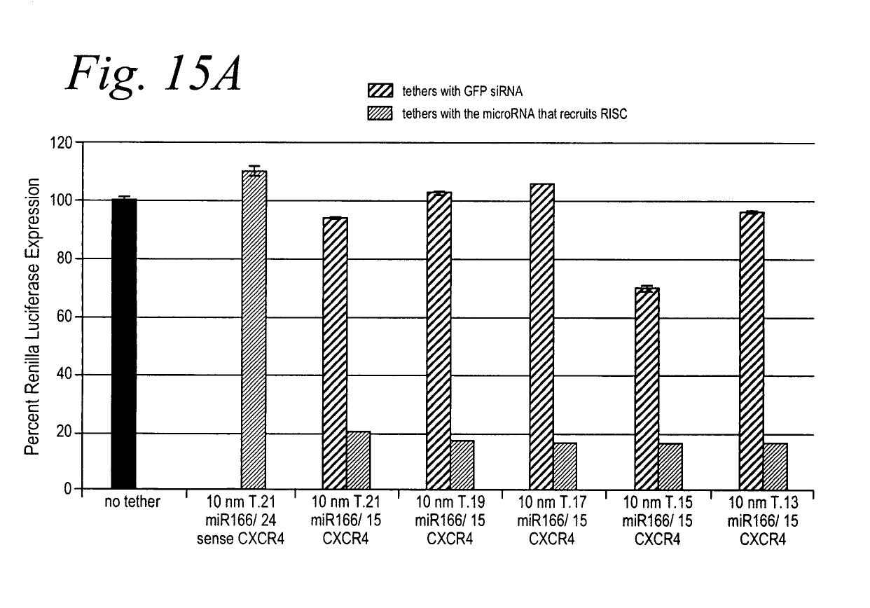

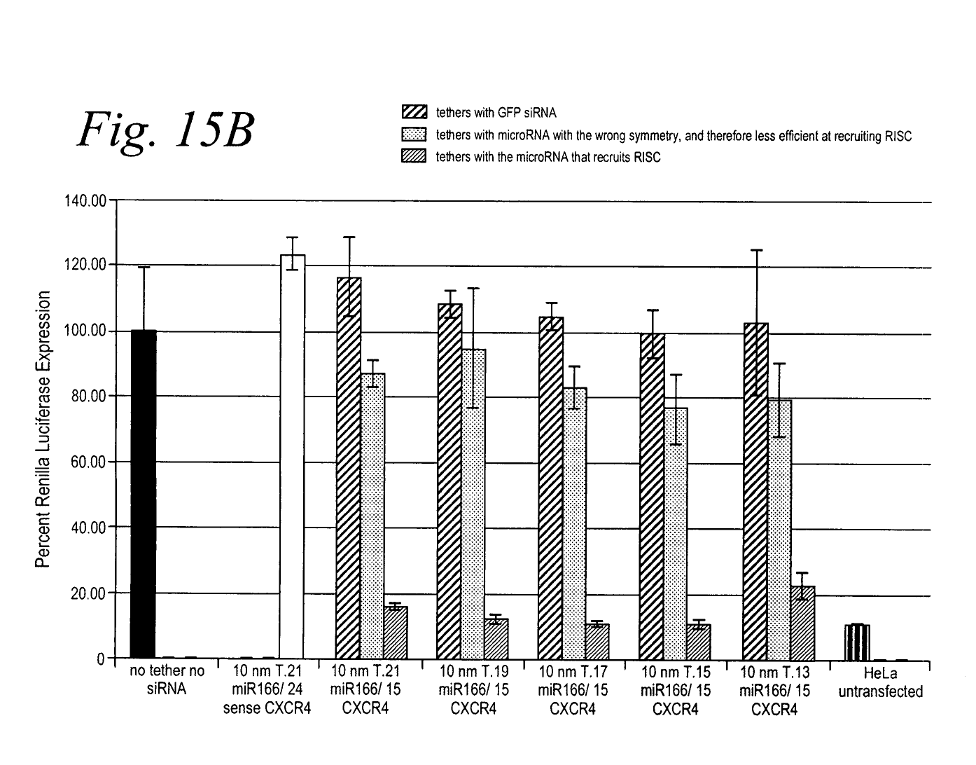

The results of the truncations of the miRNA recruiting moiety are shown in FIG. 15A. For example, 10 nM T.21 miR166/15CXCR4 displays the repression of luciferase expression in both the experimental and control HeLa cultures by an RNA silencing agent having an miRNA recruiting moiety of 21 nucleotides in length and an miRNA targeting moiety of 15 nucleotides in length. Note that 10 nM 21 miR166/24 sense CXCR4 indicates yet another control where the RNA silencing agent has an miRNA recruiting moiety of 21 nucleotides in length and an miRNA targeting moiety consisting of the sense strand of the target miRNA. Accordingly, this miRNA targeting moiety is incapable of binding to the target miRNA. As shown in FIG. 15A, even miRNA recruiting moieties of 13 nucleotides in length are effective in inducing translational repression of Renilla luciferase expression.

Yet another test was conducted to analyze truncations of the miRNA recruiting moiety. The experiment was similar to the previously described experiment in that RNA silencing agents were designed with miRNA targeting moieties of 15 nucleotides in length and with miRNA recruiting moieties with variable lengths ranging from 13 to 21 nucleotides in length. In a control culture, HeLa cells were transfected with GFP siRNA. In one experimental culture, HeLa cells were transfected with an miR166 pair designed asymmetrically, i.e., with a mismatch. The miR166 pair was as follows:

| | |
|---|---|
| Antisense | |
| 5′ CCG GAU CAG GCU UCA UCC [symbol] AA 3′ | [SEQ ID NO: 3] |
| Sense | |
| 3′ UA GGC CUA GUC CGA AGU AGG G 5′ | [SEQ ID NO: 4] |

As shown, the antisense strand was mutated at position 19 (in bold italics) so that there was a mismatch with the first nucleotide of the sense strand. This frayed or asymmetric design compelled the sense strand into the RISC. Accordingly, because the sense miR166 strand was forced into RISC, the miRNA was unable to bind to the RNA silencing agent because the sense miR166 strand and the various miRNA recruiting moieties shared the same sequences.

In another experimental culture, HeLa cells were transfected with another miR166 pair designed asymmetically. The miR166 pair was as follows:
Antisense 5′ CCG GAU CAG GCU UCA UCC CAA 3′ [SEQ ID NO: 5]
Sense 3′ UA UGC CUA GUC CGA AGU AGG G 5′ [SEQ ID NO: 6]
   As shown, the sense strand is mutated at position 19 (in bold italics) so that there is a mismatch with the first nucleotide of the antisense strand. Unlike the prior design, this frayed or asymmetric design compelled the antisense strand into the RISC. Accordingly, the antisense strand of the miR166 was capable of binding to the various miRNA recruiting moieties so as to promote repression of luciferase expression.

The results of this experiment are shown in FIG. 15B. For example, 10 nM T.21 miR166/15CXCR4 displays the repression of luciferase expression in all three HeLa cultures by an RNA silencing agent having an miRNA recruiting moiety of 21 nucleotides in length and an miRNA targeting moiety of 15 nucleotides in length. Note that 10 nM T.21 miR166/24 sense CXCR4 indicates yet another control where the RNA silencing agent has an miRNA recruiting moiety of 21 nucleotides in length and an miRNA targeting moiety consisting of the sense strand of the target miRNA. Accordingly, this miRNA targeting moiety is incapable of binding to the target miRNA. The results as shown in FIG. 15B indicate that the design of mismatches to compel the antisense strand of the miR166 pair into RISC further enhanced the ability of the RNA-silencing agents of the invention to promote translational repression.

### Example 6

### 2′-O-Methyl Oligonucleotide Tether Designed to Recruit Endogenous miRNA let-7

For the present example, 2′-O-methyl oligonucleotides were synthesized with two functional domains: an oligonucleotide region against a 3′-UTR sequence in a luciferase reporter miRNA and a domain complementary to let-7 miRNA, an abundant and potent endogenous miRNA in HeLa cells (Hutvagner et al. (2002) Science 297:2056-2060). The luciferase miRNA was engineered to have six sites for oligonucleotide complementation, so that the proximal 5′ part of the oligonucleotide binds to these six identical 24 nucleotide sites in series. Because the oligonucleotide contained a sequence fully complementary to the let-7 miRNA, the oligonucleotide-endogenous miRNA complex was designed to attract RISC, possibly prior to attachment to the Renilla reniformis luciferase miRNA. The luciferase reporter cDNA were tested in a Drosophila embryo lysate system. Subsequently, reagents were transfected into HeLa cells with Lipofectamine 2000 and, after 24 hours, cells were harvested to test for luciferase activity by standard assays.

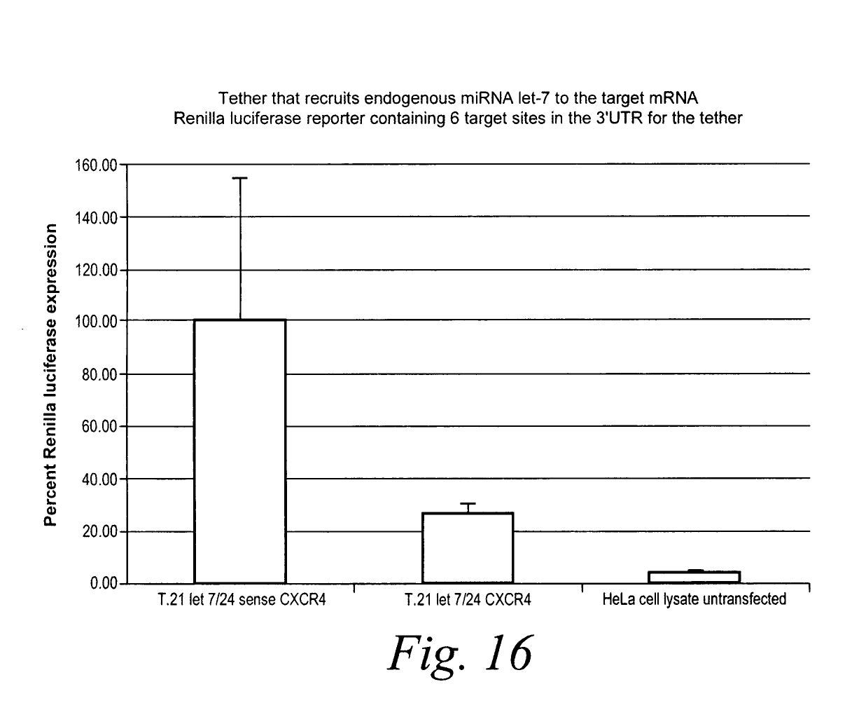

Analysis: Controls included (1) measure of luciferase expression in HeLa cells untransfected with the Renilla luciferase reporter system (shown as “HeLa cell lysate untransfected” in FIG. 5) and (2) measure of luciferase expression in HeLa cells transfected with the Renilla luciferase reporter system, but exposed to an RNA silencing agent with an miRNA targeting moiety consisting of the sense sequence and therefor, incapable of binding the target miRNA (shown as “T.21let7/24 sense CXCR4” in FIG. 16).

FIG. 16 depicts the results. As indicated therein, the RNA silencing agents of the invention were effective in harnessing endogenous miRNA let-7 and repressing luciferase expression.

The contents of all references, pending patent applications and published patents, cited throughout this application are hereby expressly incorporated by reference.

Equivalents

Those skilled in the art will recognize, or be able to ascertain using no more than routine experimentation, many equivalents to the specific embodiments of the invention described herein. Such equivalents are intended to be encompassed by the following claims.

1. An RNA-silencing agent having the following formula:
T-L-μ,
wherein T is an miRNA targeting moiety, L is a linking moiety, and μ is a miRNA recruiting moiety, forming the RNA-silencing agent.

2. An RNA silencing agent suitable for use in repressing translation of a target miRNA, comprising:
a. an miRNA targeting portion complementary to the target miRNA;
b. an miRNA recruiting portion complementary to an miRNA, wherein the miRNA is involved in repressing translation of the target miRNA; and
c. a linking portion that links the miRNA targeting portion and the miRNA recruiting portion.

3. The agent of claim 1 or 2, wherein the miRNA targeting moiety or portion is about 9 to about 24 nucleotides in length.

4. The agent of claim 1 or 2, wherein the miRNA targeting moiety or portion is 15 nucleotides in length.

5. The agent of claim 1 or 2, wherein the miRNA recruiting moiety or portion is about 13 to about 21 nucleotides in length.

6. The RNA silencing agent of claim 1 or 2, wherein the miRNA recruiting moiety or portion is about 13 or about 15 nucleotides in length.

7. The agent of claim 1 or 2, wherein the RNA is silenced via translational repression of the target miRNA.

8. The agent of claim 1 or 2, wherein the miRNA targeting moiety or portion targets an miRNA encoding a protein involved in a disease or disorder.

9. The agent of claim 1 or 2, wherein the miRNA targeting moiety or portion targets an miRNA associated with Huntington's Disease (HD).

10. The agent of claim 9, wherein the miRNA targeting moiety or portion targets an miRNA encoding huntingtin protein.

11. The agent of claim 9, wherein the miRNA targeting moiety or portion targets an miRNA encoding mutant huntingtin protein.

12. The agent of claim 1 or 2, wherein the miRNA targeting moiety targets an miRNA encoding a protein selected from the group consisting of matrix metalloproteinase 1, matrix metalloproteinase 2, matrix metalloproteinase 9, metalloelastase, CD36 receptor, tenascin-C, secreted protein acidic and rich in cysteine (SPARC), and androgen receptor gene.

13. The agent of claim 12, wherein the protein is a mutant protein.

14. The agent of claim 1 or 2, wherein the linking moiety or portion comprises a phosphodiester bond.

15. The agent of claim 1 or 2, wherein the linking moiety or portion comprises at least one modified nucleotide which increases the in vivo stability of the agent.

16. The agent of claim 15, wherein the linking moiety or portion comprises at least one 2′-O-methyl nucleotide.

17. The agent of claim 1 or 2, wherein the linking moiety or portion comprises at least one phosphorothioate nucleotide.

18. The agent of claim 1 or 2, wherein the linking moiety or portion comprises at least one 2′-O-methyl nucleotide and at least one phosphorothioate nucleotide.

19. The agent of claim 1 or 2, wherein the linking moiety or portion comprises at least one locked nucleotide.

20. The agent of claim 19, wherein the locked nucleotide is a C2′-O,C4′-ethylene-bridged nucleotide.

21. The agent of claim 1 or 2, wherein the linking moiety or portion comprises at least one sugar-modified nucleotide.

22. The agent of claim 1 or 2, wherein the linking moiety or portion comprises at least one base-modified nucleotide.

23. The agent of claim 1 or 2, wherein the linking moiety or portion comprises at least one sugar-modified nucleotide and at least one base-modified nucleotide.

24. The agent of claim 1 or 2, wherein the miRNA recruiting moiety or portion recruits an miRNA capable of inducing silencing via an RNA induced silencing complex (RISC).

25. The agent of claim 1 or 2, wherein the miRNA recruiting moiety or portion recruits an miRNA selected from Table 1.

26. The agent of claim 1 or 2, wherein the miRNA recruiting moiety or portion recruits a let-7 miRNA.

27. The agent of claim 1 or 2, wherein the miRNA recruiting moiety or portion recruits a miR124a miRNA.

28. The agent of claim 1 or 2, wherein the miRNA recruiting moiety or portion recruits a miR166 miRNA.

29. A composition comprising the RNA-silencing agent of claim 1 or 2 and a pharmaceutically acceptable carrier.

30. A method of repressing gene expression in a cell, comprising contacting a cell with the RNA-silencing agent of claim 1 or 2, under conditions such that the agent represses gene expression within the cell.

31. The method of claim 30, wherein the gene encodes a protein associated with a disease or disorder.

32. The method of claim 30, wherein the gene encodes a mutant protein.

33. The method of claim 32, wherein the gene encodes a mutant huntingtin protein.

34. The method of claim 30, wherein the cell is present in an organism.

35. A method for treating a subject having or at risk for a disease or disorder characterized or caused by the overexpression or overactivity of a cellular protein, comprising administering to the subject an effective amount of the RNA-silencing agent of claim 1 or 2, wherein the miRNA targeting moiety targets an miRNA encoding said protein.

36. A method for treating a subject having or at risk for a disease or disorder characterized or caused by the expression or activity of a mutant protein, comprising administering to the subject an effective amount of the RNA-silencing agent of claim 1 or 2, wherein the miRNA targeting moiety targets an miRNA encoding said protein.

37. The method of claim 36, wherein the disease or disorder is characterized or caused by a gain-of-function mutant protein.

38. The method of claim 36, wherein the disease is Huntington's Disease (HD).

39. The method of claim 36, wherein the protein is mutant huntingtin protein.

40. Use of the agent of claim 1 or 2 in the manufacture of a medicament for repressing mutant gene expression.


SEQUENCE LISTING (107 sequences)

SEQ ID NO: 1 - protein, 3144 aa, Homo sapiens
MATLEKLMKAFESLKSFQQQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 2 - DNA, 13672 bp, Homo sapiens
ttgctgtgtgaggcagaacctgcgggggcaggggcgggctggttccctggccagccattggcagagtccgcaggctagggctgtcaatcatgctggccggcgtggccccgcctccgccggcgcggccccgcctccgccggcgcacgtctgggacgcaaggcgccgtgggggctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccaaagaaagaactttcagctaccaagaaagaccgtgtgaatcattgtctgacaatatgtgaaaacatagtggcacagtctgtcagaaattctccagaatttcagaaacttctgggcatcgctatggaactttttctgctgtgcagtgatgacgcagagtcagatgtcaggatggtggctgacgaatgcctcaacaaagttatcaaagctttgatggattctaatcttccaaggttacagctcgagctctataaggaaattaaaaagaatggtgcccctcggagtttgcgtgctgccctgtggaggtttgctgagctggctcacctggttcggcctcagaaatgcaggccttacctggtgaaccttctgccgtgcctgactcgaacaagcaagagacccgaagaatcagtccaggagaccttggctgcagctgttcccaaaattatggcttcttttggcaattttgcaaatgacaatgaaattaaggttttgttaaaggccttcatagcgaacctgaagtcaagctcccccaccattcggcggacagcggctggatcagcagtgagcatctgccagcactcaagaaggacacaatatttctatagttggctactaaatgtgctcttaggcttactcgttcctgtcgaggatgaacactccactctgctgattcttggcgtgctgctcaccctgaggtatttggtgcccttgctgcagcagcaggtcaaggacacaagcctgaaaggcagcttcggagtgacaaggaaagaaatggaagtctctccttctgcagagcagcttgtccaggtttatgaactgacgttacatcatacacagcaccaagaccacaatgttgtgaccggagccctggagctgttgcagcagctcttcagaacgcctccacccgagcttctgcaaaccctgaccgcagtcgggggcattgggcagctcaccgctgctaaggaggagtctggtggccgaagccgtagtgggagtattgtggaacttatagctggagggggttcctcatgcagccctgtcctttcaagaaaacaaaaaggcaaagtgctcttaggagaagaagaagccttggaggatgactctgaatcgagatcggatgtcagcagctctgccttaacagcctcagtgaaggatgagatcagtggagagctggctgcttcttcaggggtttccactccagggtcagcaggtcatgacatcatcacagaacagccacggtcacagcacacactgcaggcggactcagtggatctggccagctgtgacttgacaagctctgccactgatggggatgaggaggatatcttgagccacagctccagccaggtcagcgccgtcccatctgaccctgccatggacctgaatgatgggacccaggcctcgtcgcccatcagcgacagctcccagaccaccaccgaagggcctgattcagctgttaccccttcagacagttctgaaattgtgttagacggtaccgacaaccagtatttgggcctgcagattggacagccccaggatgaagatgaggaagccacaggtattcttcctgatgaagcctcggaggccttcaggaactcttccatggcccttcaacaggcacatttattgaaaaacatgagtcactgcaggcagccttctgacagcagtgttgataaatttgtgttgagagatgaagctactgaaccgggtgatcaagaaaacaagccttgccgcatcaaaggtgacattggacagtccactgatgatgactctgcacctcttgtccattgtgtccgccttttatctgcttcgtttttgctaacagggggaaaaaatgtgctggttccggacagggatgtgagggtcagcgtgaaggccctggccctcagctgtgtgggagcagctgtggccctccacccggaatctttcttcagcaaactctataaagttcctcttgacaccacggaataccctgaggaacagtatgtctcagacatcttgaactacatcgatcatggagacccacaggttcgaggagccactgccattctctgtgggaccctcatctgctccatcctcagcaggtcccgcttccacgtgggagattggatgggcaccattagaaccctcacaggaaatacattttctttggcggattgcattcctttgctgcggaaaacactgaaggatgagtcttctgttacttgcaagttagcttgtacagctgtgaggaactgtgtcatgagtctctgcagcagcagctacagtgagttaggactgcagctgatcatcgatgtgctgactctgaggaacagttcctattggctggtgaggacagagcttctggaaacccttgcagagattgacttcaggctggtgagctttttggaggcaaaagcagaaaacttacacagaggggctcatcattatacagggcttttaaaactgcaagaacgagtgctcaataatgttgtcatccatttgcttggagatgaagaccccagggtgcgacatgttgccgcagcatcactaattaggcttgtcccaaagctgttttataaatgtgaccaaggacaagctgatccagtagtggccgtggcaagagatcaaagcagtgtttacctgaaacttctcatgcatgagacgcagcctccatctcatttctccgtcagcacaataaccagaatatatagaggctataacctactaccaagcataacagacgtcactatggaaaataacctttcaagagttattgcagcagtttctcatgaactaatcacatcaaccaccagagcactcacatttggatgctgtgaagctttgtgtcttctttccactgccttcccagtttgcatttggagtttaggttggcactgtggagtgcctccactgagtgcctcagatgagtctaggaagagctgtaccgttgggatggccacaatgattctgaccctgctctcgtcagcttggttcccattggatctctcagcccatcaagatgctttgattttggccggaaacttgcttgcagccagtgctcccaaatctctgagaagttcatgggcctctgaagaagaagccaacccagcagccaccaagcaagaggaggtctggccagccctgggggaccgggccctggtgcccatggtggagcagctcttctctcacctgctgaaggtgattaacatttgtgcccacgtcctggatgacgtggctcctggacccgcaataaaggcagccttgccttctctaacaaaccccccttctctaagtcccatccgacgaaaggggaaggagaaagaaccaggagaacaagcatctgtaccgttgagtcccaagaaaggcagtgaggccagtgcagcttctagacaatctgatacctcaggtcctgttacaacaagtaaatcctcatcactggggagtttctatcatcttccttcatacctcaaactgcatgatgtcctgaaagctacacacgctaactacaaggtcacgctggatcttcagaacagcacggaaaagtttggagggtttctccgctcagccttggatgttctttctcagatactagagctggccacactgcaggacattgggaagtgtgttgaagagatcctaggatacctgaaatcctgctttagtcgagaaccaatgatggcaactgtttgtgttcaacaattgttgaagactctctttggcacaaacttggcctcccagtttgatggcttatcttccaaccccagcaagtcacaaggccgagcacagcgccttggctcctccagtgtgaggccaggcttgtaccactactgcttcatggccccgtacacccacttcacccaggccctcgctgacgccagcctgaggaacatggtgcaggcggagcaggagaacgacacctcgggatggtttgatgtcctccagaaagtgtctacccagttgaagacaaacctcacgagtgtcacaaagaaccgtgcagataagaatgctattcataatcacattcgtttgtttgaacctcttgttataaaagctttaaaacagtacacgactacaacatgtgtgcagttacagaagcaggttttagatttgctggcgcagctggttcagttacgggttaattactgtcttctggattcagatcaggtgtttattggctttgtattgaaacagtttgaatacattgaagtgggccagttcagggaatcagaggcaatcattccaaacatctttttcttcttggtattactatcttatgaacgctatcattcaaaacagatcattggaattcctaaaatcattcagctctgtgatggcatcatggccagtggaaggaaggctgtgacacatgccataccggctctgcagcccatagtccacgacctctttgtattaagaggaacaaataaagctgatgcaggaaaagagcttgaaacccaaaaagaggtggtggtgtcaatgttactgagactcatccagtaccatcaggtgttggagatgttcattcttgtcctgcagcagtgccacaaggagaatgaagacaagtggaagcgactgtctcgacagatagctgacatcatcctcccaatgttagccaaacagcagatgcacattgactctcatgaagcccttggagtgttaaatacattatttgagattttggccccttcctccctccgtccggtagacatgcttttacggagtatgttcgtcactccaaacacaatggcgtccgtgagcactgttcaactgtggatatcgggaattctggccattttgagggttctgatttcccagtcaactgaagatattgttctttctcgtattcaggagctctccttctctccgtatttaatctcctgtacagtaattaataggttaagagatggggacagtacttcaacgctagaagaacacagtgaagggaaacaaataaagaatttgccagaagaaacattttcaaggtttctattacaactggttggtattcttttagaagacattgttacaaaacagctgaaggtggaaatgagtgagcagcaacatactttctattgccaggaactaggcacactgctaatgtgtctgatccacatcttcaagtctggaatgttccggagaatcacagcagctgccactaggctgttccgcagtgatggctgtggcggcagtttctacaccctggacagcttgaacttgcgggctcgttccatgatcaccacccacccggccctggtgctgctctggtgtcagatactgctgcttgtcaaccacaccgactaccgctggtgggcagaagtgcagcagaccccgaaaagacacagtctgtccagcacaaagttacttagtccccagatgtctggagaagaggaggattctgacttggcagccaaacttggaatgtgcaatagagaaatagtacgaagaggggctctcattctcttctgtgattatgtctgtcagaacctccatgactccgagcacttaacgtggctcattgtaaatcacattcaagatctgatcagcctttcccacgagcctccagtacaggacttcatcagtgccgttcatcggaactctgctgccagcggcctgttcatccaggcaattcagtctcgttgtgaaaacctttcaactccaaccatgctgaagaaaactcttcagtgcttggaggggatccatctcagccagtcgggagctgtgctcacgctgtatgtggacaggcttctgtgcacccctttccgtgtgctggctcgcatggtcgacatccttgcttgtcgccgggtagaaatgcttctggctgcaaatttacagagcagcatggcccagttgccaatggaagaactcaacagaatccaggaataccttcagagcagcgggctcgctcagagacaccaaaggctctattccctgctggacaggtttcgtctctccaccatgcaagactcacttagtccctctcctccagtctcttcccacccgctggacggggatgggcacgtgtcactggaaacagtgagtccggacaaagactggtacgttcatcttgtcaaatcccagtgttggaccaggtcagattctgcactgctggaaggtgcagagctggtgaatcggattcctgctgaagatatgaatgccttcatgatgaactcggagttcaacctaagcctgctagctccatgcttaagcctagggatgagtgaaatttctggtggccagaagagtgccctttttgaagcagcccgtgaggtgactctggcccgtgtgagcggcaccgtgcagcagctccctgctgtccatcatgtcttccagcccgagctgcctgcagagccggcggcctactggagcaagttgaatgatctgtttggggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgaggccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccgttgcagtgcagcctggagagcagcttcttagtccagaaagaaggacaaataccccaaaagccatcagcgaggaggaggaggaagtagatccaaacacacagaatcctaagtatatcactgcagcctgtgagatggtggcagaaatggtggagtctctgcagtcggtgttggccttgggtcataaaaggaatagcggcgtgccggcgtttctcacgccattgctcaggaacatcatcatcagcctggcccgcctgccccttgtcaacagctacacacgtgtgcccccactggtgtggaagcttggatggtcacccaaaccgggaggggattttggcacagcattccctgagatccccgtggagttcctccaggaaaaggaagtctttaaggagttcatctaccgcatcaacacactaggctggaccagtcgtactcagtttgaagaaacttgggccaccctccttggtgtcctggtgacgcagcccctcgtgatggagcaggaggagagcccaccagaagaagacacagagaggacccagatcaacgtcctggccgtgcaggccatcacctcactggtgctcagtgcaatgactgtgcctgtggccggcaacccagctgtaagctgcttggagcagcagccccggaacaagcctctgaaagctctcgacaccaggtttgggaggaagctgagcattatcagagggattgtggagcaagagattcaagcaatggtttcaaagagagagaatattgccacccatcatttatatcaggcatgggatcctgtcccttctctgtctccggctactacaggtgccctcatcagccacgagaagctgctgctacagatcaaccccgagcgggagctggggagcatgagctacaaactcggccaggtgtccatacactccgtgtggctggggaacagcatcacacccctgagggaggaggaatgggacgaggaagaggaggaggaggccgacgcccctgcaccttcgtcaccacccacgtctccagtcaactccaggaaacaccgggctggagttgacatccactcctgttcgcagtttttgcttgagttgtacagccgctggatcctgccgtccagctcagccaggaggaccccggccatcctgatcagtgaggtggtcagatcccttctagtggtctcagacttgttcaccgagcgcaaccagtttgagctgatgtatgtgacgctgacagaactgcgaagggtgcacccttcagaagacgagatcctcgctcagtacctggtgcctgccacctgcaaggcagctgccgtccttgggatggacaaggccgtggcggagcctgtcagccgcctgctggagagcacgctcaggagcagccacctgcccagcagggttggagccctgcacggcgtcctctatgtgctggagtgcgacctgctggacgacactgccaagcagctcatcccggtcatcagcgactatctcctctccaacctgaaagggatcgcccactgcgtgaacattcacagccagcagcacgtactggtcatgtgtgccactgcgttttacctcattgagaactatcctctggacgtagggccggaattttcagcatcaataatacagatgtgtggggtgatgctgtctggaagtgaggagtccaccccctccatcatttaccactgtgccctcagaggcctggagcgcctcctgctctctgagcagctctcccgcctggatgcagaatcgctggtcaagctgagtgtggacagagtgaacgtgcacagcccgcaccgggccatggcggctctgggcctgatgctcacctgcatgtacacaggaaaggagaaagtcagtccgggtagaacttcagaccctaatcctgcagcccccgacagcgagtcagtgattgttgctatggagcgggtatctgttctttttgataggatcaggaaaggctttccttgtgaagccagagtggtggccaggatcctgccccagtttctagacgacttcttcccaccccaggacatcatgaacaaagtcatcggagagtttctgtccaaccagcagccatacccccagttcatggccaccgtggtgtataaggtgtttcagactctgcacagcaccgggcagtcgtccatggtccgggactgggtcatgctgtccctctccaacttcacgcagagggccccggtcgccatggccacgtggagcctctcctgcttctttgtcagcgcgtccaccagcccgtgggtcgcggcgatcctcccacatgtcatcagcaggatgggcaagctggagcaggtggacgtgaaccttttctgcctggtcgccacagacttctacagacaccagatagaggaggagctcgaccgcagggccttccagtctgtgcttgaggtggttgcagccccaggaagcccatatcaccggctgctgacttgtttacgaaatgtccacaaggtcaccacctgctgagcgccatggtgggagagactgtgaggcggcagctggggccggagcctttggaagtctgtgcccttgtgccctgcctccaccgagccagcttggtccctatgggcttccgcacatgccgcgggcggccaggcaacgtgcgtgtctctgccatgtggcagaagtgctctttgtggcagtggccaggcagggagtgtctgcagtcctggtggggctgagcctgaggccttccagaaagcaggagcagctgtgctgcaccccatgtgggtgaccaggtcctttctcctgatagtcacctgctggttgttgccaggttgcagctgctcttgcatctgggccagaagtcctccctcctgcaggctggctgttggcccctctgctgtcctgcagtagaaggtgccgtgagcaggctttgggaacactggcctgggtctccctggtggggtgtgcatgccacgccccgtgtctggatgcacagatgccatggcctgtgctgggccagtggctgggggtgctagacacccggcaccattctcccttctctcttttcttctcaggatttaaaatttaattatatcagtaaagagattaattttaacgaactctttctatgcccgtgtaaagtatgtgaatcgcaaggcctgtgctgcatgcgacagcgtccggggtggtggacagggcccccggccacgctccctctcctgtagccactggcatagccctcctgagcacccgctgacatttccgttgtacatgttcctgtttatgcattcacaaggtgactgggatgtagagaggcgttagtgggcaggtggccacagcaggactgaggacaggcccccattatcctaggggtgcgctcaactgcagcccctcctcctcgggcacagacgactgtcgttctccacccaccagtcagggacagcagcctccctgtcactcagctgagaaggccagccctccctggctgtgagcagcctccactgtgtccagagacatgggcctcccactcctgttccttgctagccctggggtggcgtctgcctaggagctggctggcaggtgttgggacctgctgctccatggatgcatgccctaagagtgtcactgagctgtgttttgtctgagcctctctcggtcaacagcaaagcttggtgtcttggcactgttagtgacagagcccagcatcccttctgcccccgttccagctgacatcttgcacggtgaccccttttagtcaggagagtgcagatctgtgctcatcggagactgccccacggccctgtcagagccgccactcctatccccaggacaggtccctggaccagcctcctgtttgcaggcccagaggagccaagtcattaaaatggaagtggattctggatggccgggctgctgctgatgtaggagctggatttgggagctctgcttgccgactggctgtgagacgaggcaggggctctgcttcctcagccctagaggcgagccaggcaaggttggcgactgtcatgtggcttggtttggtcatgcccgtcgatgttttgggtattgaatgtggtaagtggaggaaatgttggaactctgtgcaggtgctgccttgagacccccaagcttccacctgtccctctcctatgtggcagctggggagcagctgagatgtggacttgtatgctgcccacatacgtgagggggagctgaaagggagcccctgctcaaagggagcccctcctctgagcagcctctgccaggcctgtatgaggcttttcccaccagctcccaacagaggcctcccccagccaggaccacctcgtcctcgtggcggggcagcaggagcggtagaaaggggtccgatgtttgaggaggcccttaagggaagctactgaattataacacgtaagaaaatcaccattcttccgtattggttgggggctcctgtttctcatcctagctttttcctggaaaagcccgctagaaggtttgggaacgaggggaaagttctcagaactgttgctgctccccacccgcctcccgcctcccccgcaggttatgtcagcagctctgagacagcagtatcacaggccagatgttgttcctggctagatgtttacatttgtaagaaataacactgtgaatgtaaaacagagccattcccttggaatgcatatcgctgggctcaacatagagtttgtcttcctcttgtttacgacgtgatctaaaccagtccttagcaaggggctcagaacaccccgctctggcagtaggtgtcccccacccccaaagacctgcctgtgtgctccggagatgaatatgagctcattagtaaaaatgacttcacccacgcatatacataaagtatccatgcatgtgcatatagacacatctataattttacacacacacctctcaagacggagatgcatggcctctaagagtgcccgtgtcggttcttcctggaagttgactttccttagacccgccaggtcaagttagccgcgtgacggacatccaggcgtgggacgtggtcagggcagggctcattcattgcccactaggatcccactggcgaagatggtctccatatcagctctctgcagaagggaggaagactttatcatgttcctaaaaatctgtggcaagcacccatcgtattatccaaattttgttgcaaatgtgattaatttggttgtcaagttttgggggtgggctgtggggagattgcttttgttttcctgctggtaatatcgggaaagattttaatgaaaccagggtagaattgtttggcaatgcactgaagcgtgtttctttcccaaaatgtgcctcccttccgctgcgggcccagctgagtctatgtaggtgatgtttccagctgccaagtgctctttgttactgtccaccctcatttctgccagcgcatgtgtcctttcaaggggaaaatgtgaagctgaaccccctccagacacccagaatgtagcatctgagaaggccctgtgccctaaaggacacccctcgcccccatcttcatggagggggtcatttcagagccctcggagccaatgaacagctcctcctcttggagctgagatgagccccacgtggagctcgggacggatagtagacagcaataactcggtgtgtggccgcctggcaggtggaacttcctcccgttgcggggtggagtgaggttagttctgtgtgtctggtgggtggagtcaggcttctcttgctacctgtgagcatccttcccagcagacatcctcatcgggctttgtccctcccccgcttcctccctctgcggggaggacccgggaccacagctgctggccagggtagacttggagctgtcctccagaggggtcacgtgtaggagtgagaagaaggaagatcttgagagctgctgagggaccttggagagctcaggatggctcagacgaggacactcgcttgccgggcctggccctcctgggaaggagggagctgctcagaatgccgcatgacaactgaaggcaacctggaaggttcagggcccgctcttcccccatgtgcctgtcacgctctggtgcagtcaaaggaacgccttcccctcagttgtttctaagagcagagtctcccgctgcaatctgggtggtaactgccagccttggaggatcgtggccaacgtggacctgcctacggagggtgggctctgacccaagtggggcctccttgcccaggtctcactgctttgcaccgtggtcagagggactgtcagctgagcttgagctcccctggagccagcagggctgtgatgggcgagtcccggagccccacccagacctgaatgcttctgagagcaaagggaaggactgacgagagatgtatatttaattttttaactgctgcaaacattgtacatccaaattaaagggaaaaaatggaaaccatcaat

SEQ ID NO: 3 - RNA, 21 nt, Artificial Sequence
  Description of Artificial Sequence Synthetic oligonucleotide
ccggaucaggcuucauccaaa

SEQ ID NO: 4 - RNA, 21 nt, Artificial Sequence
  Description of Artificial Sequence Synthetic oligonucleotide
gggaugaagccugauccggau

SEQ ID NO: 5 - RNA, 21 nt, Artificial Sequence
  Description of Artificial Sequence Synthetic oligonucleotide
ccggaucaggcuucaucccaa

SEQ ID NO: 6 - RNA, 21 nt, Artificial Sequence
  Description of Artificial Sequence Synthetic oligonucleotide
gggaugaagccugauccguau

SEQ ID NO: 7 - RNA, 21 nt, Homo sapiens
uggaagacuagugauuuuguu

SEQ ID NO: 8 - RNA, 22 nt, Homo sapiens
ugagguaguagauuguauaguu

SEQ ID NO: 9 - RNA, 21 nt, Homo sapiens
ugagguaggagguuguauagu

SEQ ID NO: 10 - RNA, 22 nt, Homo sapiens
ugagguaguagguuguauaguu

SEQ ID NO: 11 - RNA, 22 nt, Homo sapiens
ugagguaguagguugugugguu

SEQ ID NO: 12 - RNA, 22 nt, Homo sapiens
ugagguaguagguuguaugguu

SEQ ID NO: 13 - RNA, 21 nt, Homo sapiens
agagguaguagguugcauagu

SEQ ID NO: 14 - RNA, 23 nt, Homo sapiens
uacccuguagauccgaauuugug

SEQ ID NO: 15 - RNA, 22 nt, Homo sapiens
uacccuguagaaccgaauuugu

SEQ ID NO: 16 - RNA, 22 nt, Homo sapiens
uagcagcacauaaugguuugug

SEQ ID NO: 17 - RNA, 22 nt, Homo sapiens
uagcagcacguaaauauuggcg

SEQ ID NO: 18 - RNA, 20 nt, Homo sapiens
acugcagugaaggcacuugu

SEQ ID NO: 19 - RNA, 22 nt, Homo sapiens
uaaggugcaucuagugcagaua

SEQ ID NO: 20 - RNA, 23 nt, Homo sapiens
ugugcaaaucuaugcaaaacuga

SEQ ID NO: 21 - RNA, 23 nt, Homo sapiens
ugugcaaauccaugcaaaacuga

SEQ ID NO: 22 - RNA, 22 nt, Homo sapiens
uaaagugcuuauagugcaggua

SEQ ID NO: 23 - RNA, 22 nt, Homo sapiens
uagcuuaucagacugauguuga

SEQ ID NO: 24 - RNA, 22 nt, Homo sapiens
aagcugccaguugaagaacugu

SEQ ID NO: 25 - RNA, 21 nt, Homo sapiens
aucacauugccagggauuucc

SEQ ID NO: 26 - RNA, 22 nt, Homo sapiens
uggcucaguucagcaggaacag

SEQ ID NO: 27 - RNA, 22 nt, Homo sapiens
cauugcacuugucucggucuga

SEQ ID NO: 28 - RNA, 22 nt, Homo sapiens
uucaaguaauccaggauaggcu

SEQ ID NO: 29 - RNA, 21 nt, Homo sapiens
uucaaguaauucaggauaggu

SEQ ID NO: 30 - RNA, 22 nt, Homo sapiens
uucacaguggcuaaguuccgcc

SEQ ID NO: 31 - RNA, 22 nt, Homo sapiens
aaggagcucacagucuauugag

SEQ ID NO: 32 - RNA, 22 nt, Homo sapiens
cuagcaccaucugaaaucgguu

SEQ ID NO: 33 - RNA, 23 nt, Homo sapiens
uguaaacauccuacacucucagc

SEQ ID NO: 34 - RNA, 22 nt, Homo sapiens
uguaaacauccccgacuggaag

SEQ ID NO: 35 - RNA, 23 nt, Homo sapiens
uguaaacauccucgacuggaagc

SEQ ID NO: 36 - RNA, 22 nt, Homo sapiens
cuuucagucggauguuugcagc

SEQ ID NO: 37 - RNA, 21 nt, Homo sapiens
ggcaagaugcuggcauagcug

SEQ ID NO: 38 - RNA, 21 nt, Homo sapiens
uauugcacauuacuaaguugc

SEQ ID NO: 39 - RNA, 19 nt, Homo sapiens
gugcauuguaguugcauug

SEQ ID NO: 40 - RNA, 22 nt, Homo sapiens
uggcagugucuuagcugguugu

SEQ ID NO: 41 - RNA, 24 nt, Homo sapiens
caaagugcuuacagugcagguagu

SEQ ID NO: 42 - RNA, 22 nt, Homo sapiens
uauugcacuugucccggccugu

SEQ ID NO: 43 - RNA, 22 nt, Homo sapiens
aaagugcuguucgugcagguag

SEQ ID NO: 44 - RNA, 22 nt, Homo sapiens
uucaacggguauuuauugagca

SEQ ID NO: 45 - RNA, 22 nt, Homo sapiens
uuuggcacuagcacauuuuugc

SEQ ID NO: 46 - RNA, 22 nt, Homo sapiens
ugagguaguaaguuguauuguu

SEQ ID NO: 47 - RNA, 22 nt, Homo sapiens
aacccguagauccgaucuugug

SEQ ID NO: 48 - RNA, 22 nt, Homo sapiens
aacccguagauccgaacuugug

SEQ ID NO: 49 - RNA, 22 nt, Homo sapiens
uacaguacugugauaacugaag

SEQ ID NO: 50 - RNA, 20 nt, Homo sapiens
uagcaccauuugaaaucagu

SEQ ID NO: 51 - RNA, 23 nt, Homo sapiens
agcaacauuguacagggcuauga

SEQ ID NO: 52 - RNA, 23 nt, Homo sapiens
agcagcauuguacagggcuauga

SEQ ID NO: 53 - RNA, 22 nt, Homo sapiens
ucaacaucagucugauaagcua

SEQ ID NO: 54 - RNA, 20 nt, Homo sapiens
ucaaaugcucagacuccugu

SEQ ID NO: 55 - RNA, 24 nt, Homo sapiens
aaaagugcuuacagugcagguagc

SEQ ID NO: 56 - RNA, 23 nt, Homo sapiens
agcagcauuguacagggcuauca

SEQ ID NO: 57 - RNA, 20 nt, Homo sapiens
uuaaggcacgcggugaaugc

SEQ ID NO: 58 - RNA, 18 nt, Homo sapiens
ucuacagugcacgugucu

SEQ ID NO: 59 - RNA, 20 nt, Homo sapiens
guguguggaaaugcuucugc

SEQ ID NO: 60 - RNA, 22 nt, Homo sapiens
ucagugcacuacagaacuuugu

SEQ ID NO: 61 - RNA, 22 nt, Homo sapiens
aacauucaaccugucggugagu

SEQ ID NO: 62 - RNA, 22 nt, Homo sapiens
accaucgaccguugauuguacc

SEQ ID NO: 63 - RNA, 23 nt, Homo sapiens
aacauucaacgcugucggugagu

SEQ ID NO: 64 - RNA, 21 nt, Homo sapiens
ugguucuagacuugccaacua

SEQ ID NO: 65 - RNA, 23 nt, Homo sapiens
uauggcacugguagaauucacug

SEQ ID NO: 66 - RNA, 21 nt, Homo sapiens
ucgugucuuguguugcagccg

SEQ ID NO: 67 - RNA, 21 nt, Homo sapiens
cugaccuaugaauugacagcc

SEQ ID NO: 68 - RNA, 22 nt, Homo sapiens
uagguaguuucauguuguuggg

SEQ ID NO: 69 - RNA, 21 nt, Homo sapiens
uagguaguuucauguuguugg

SEQ ID NO: 70 - RNA, 22 nt, Homo sapiens
uucaccaccuucuccacccagc

SEQ ID NO: 71 - RNA, 19 nt, Homo sapiens
gguccagaggggagauagg

SEQ ID NO: 72 - RNA, 23 nt, Homo sapiens
cccaguguucagacuaccuguuc

SEQ ID NO: 73 - RNA, 23 nt, Homo sapiens
cccaguguuuagacuaucuguuc

SEQ ID NO: 74 - RNA, 22 nt, Homo sapiens
cccaguguucagacuaccuguu

SEQ ID NO: 75 - RNA, 24 nt, Homo sapiens
cucuaauacugccugguaaugaug

SEQ ID NO: 76 - RNA, 22 nt, Homo sapiens
gugaaauguuuaggaccacuag

SEQ ID NO: 77 - RNA, 22 nt, Homo sapiens
uucccuuugucauccuaugccu

SEQ ID NO: 78 - RNA, 22 nt, Homo sapiens
uccuucauuccaccggagucug

SEQ ID NO: 79 - RNA, 22 nt, Homo sapiens
auaagacgagcaaaaagcuugu

SEQ ID NO: 80 - RNA, 21 nt, Homo sapiens
cugugcgugugacagcggcug

SEQ ID NO: 81 - RNA, 22 nt, Homo sapiens
uucccuuugucauccuucgccu

SEQ ID NO: 82 - RNA, 21 nt, Homo sapiens
uaacagucuccagucacggcc

SEQ ID NO: 83 - RNA, 24 nt, Homo sapiens
aacauucauugcugucgguggguu

SEQ ID NO: 84 - RNA, 21 nt, Homo sapiens
acagcaggcacagacaggcag

SEQ ID NO: 85 - RNA, 21 nt, Homo sapiens
augaccuaugaauugacagac

SEQ ID NO: 86 - RNA, 21 nt, Homo sapiens
uaaucucagcuggcaacugug

SEQ ID NO: 87 - RNA, 24 nt, Homo sapiens
uacugcaucaggaacugauuggau

SEQ ID NO: 88 - RNA, 21 nt, Homo sapiens
uugugcuugaucuaaccaugu

SEQ ID NO: 89 - RNA, 21 nt, Homo sapiens
ugauuguccaaacgcaauucu

SEQ ID NO: 90 - RNA, 21 nt, Homo sapiens
ccacaccguaucugacacuuu

SEQ ID NO: 91 - RNA, 23 nt, Homo sapiens
agcuacauugucugcuggguuuc

SEQ ID NO: 92 - RNA, 24 nt, Homo sapiens
agcuacaucuggcuacugggucuc

SEQ ID NO: 93 - RNA, 21 nt, Homo sapiens
ugucaguuugucaaauacccc

SEQ ID NO: 94 - RNA, 23 nt, Homo sapiens
caagucacuagugguuccguuua

SEQ ID NO: 95 - RNA, 46 nt, Artificial Sequence
  Description of Artificial Sequence Synthetic oligonucleotide
uugggaugaagccugauccggccgguguuagcuggagugaaaacuu

SEQ ID NO: 96 - RNA, 21 nt, Artificial Sequence
  Description of Artificial Sequence Synthetic oligonucleotide
ccggaucaggcuucaucccaa

SEQ ID NO: 97 - RNA, 21 nt, Artificial Sequence
  Description of Artificial Sequence Synthetic oligonucleotide
gggaugaagccugauccguau

SEQ ID NO: 98 - RNA, 46 nt, Artificial Sequence
  Description of Artificial Sequence Synthetic oligonucleotide
ccggaucaggcuucaucccaaccgguguuagcuggagugaaaacuu

SEQ ID NO: 99 - RNA, 20 nt, Artificial Sequence
  Description of Artificial Sequence Synthetic oligonucleotide
gggaugaagccugauccuau

SEQ ID NO: 100 - RNA, 21 nt, Artificial Sequence
  Description of Artificial Sequence Synthetic oligonucleotide
ccggaucaggcuucaucccaa

SEQ ID NO: 101 - DNA, 24 bp, Artificial Sequence
  Description of Artificial Sequence Synthetic oligonucleotide
aagttttcacaaagctaacaccgg

SEQ ID NO: 102 - DNA, 21 bp, Artificial Sequence
  Description of Artificial Sequence Synthetic oligonucleotide
uguuagcuggagugaaaactt

SEQ ID NO: 103 - RNA, 21 nt, Artificial Sequence
  Description of Artificial Sequence Synthetic oligonucleotide
guuuucacuccagcuaacaca

SEQ ID NO: 104 - DNA, 24 bp, Artificial Sequence
  Description of Artificial Sequence Synthetic oligonucleotide
aagttttcacaaagctaacaccgg

SEQ ID NO: 105 - RNA, 21 nt, Artificial Sequence
  Description of Artificial Sequence Synthetic oligonucleotide
guguuagcuuugugaaaacuu

SEQ ID NO: 106 - RNA, 21 nt, Artificial Sequence
  Description of Artificial Sequence Synthetic oligonucleotide
guuuucacaaagcuaacaccg

SEQ ID NO: 107 - RNA, 55 nt, Artificial Sequence
  Description of Artificial Sequence Synthetic 2′-O-methyl oligonucleotide
ucuucacuauacaaccuacuaccucaaccuuccgguguuagcuuugugaaaacuu